(19) United States
(12) Patent Application Publication
(10) Pub. No.: US 2026/0345328 A1
(43) Pub. Date: Nov. 5, 2026

(54) HEART VALVE SEALING DEVICES AND DELIVERY DEVICES THEREFOR

(71) Applicant: Edwards Lifesciences Corporation, Irvine, CA (US)

(72) Inventors: Minh T. Ma, Santa Ana, CA (US); Sergio Delgado, Irvine, CA (US)

(21) Appl. No.: 19/739,357

(22) Filed: Jul. 13, 2026

Related U.S. Application Data
(63) Continuation of application No. 17/845,364, filed on Jun. 21, 2022, now Pat. No. 12,702,554.
Application No. 17/845,364 is a continuation of application No. PCT/US2020/065383, filed on Dec. 16, 2020.
(60) Provisional application No. 62/953,098, filed on Dec. 23, 2019.

(51) Int. Cl. A61F 2/24

(52) U.S. Cl. CPC A61F 2/246; A61F 2/2454; A61F 2/2463; A61F 2220/0008

(57) ABSTRACT

A valve repair device includes a pair of anchors and at least one leak inhibitor. The device can also include a spacer or coaptation element. The pair of anchors can be coupled to the spacer or coaptation element. The pair of anchors are movable between an open position and a closed position and are configured to attach the valve repair device to the native valve of the patient. The at least one leak inhibitor extends between the pair of anchors and is configured to inhibit retrograde blood flow through the device, for example, between one or more openings between leaflet portions that are in the anchors.

### RELATED APPLICATIONS

The present application is a continuation of United States Patent Application Serial No. 17/845,364, filed on Jun. 21, 2022, which is a continuation of Patent Cooperation Treaty Application No. PCT/US/2020/065383, filed on Dec. 16, 2020, which claims the benefit of U.S. provisional application no. 62/953,098 filed on Dec. 23, 2019, which are all incorporated herein by reference in their entireties for all purposes.

### BACKGROUND OF THE INVENTION

The native heart valves (i.e., the aortic, pulmonary, tricuspid, and mitral valves) serve critical functions in assuring the forward flow of an adequate supply of blood through the cardiovascular system. These heart valves can be damaged, and thus rendered less effective, for example, by congenital malformations, inflammatory processes, infectious conditions, disease, etc. Such damage to the valves can result in serious cardiovascular compromise or death. Damaged valves can be surgically repaired or replaced during open heart surgery. However, open heart surgeries are highly invasive, and complications may occur. Transvascular techniques can be used to introduce and implant prosthetic devices in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a prosthetic device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus can form a “D”-shaped, oval, or otherwise out-of-round cross-sectional shape having major and minor axes. The anterior leaflet can be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation can have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

A native valve of a patient can be repaired by attaching a spacer between leaflets of the native valve of the patient. Retrograde blood flow through gaps between the spacer and the leaflets is blocked or inhibited.

An example valve repair device includes a spacer, a pair of paddles, and at least one leak control extension. The pair of anchors (e.g., paddles, latches, clamps, grippers, fasteners, etc.) can be coupled to the spacer. The pair of anchors (e.g., a pair of paddles) are movable between an open position and a closed position and are configured to attach the valve repair device to the native valve of the patient. The at least one leak control extension extends from the spacer and is configured to block retrograde blood flow along sides of the spacer.

An example valve repair system includes a delivery sheath and a valve repair device. The valve repair device is deployable to the native valve of the patient by the delivery sheath. The valve repair device includes a spacer, a pair of paddles, and at least one leak control extension. The pair of paddles are coupled to the spacer. The pair of paddles are movable between an open position and a closed position and are configured to attach the valve repair device to the native valve of the patient. The at least one leak control extension extends from the spacer and is configured to block retrograde blood flow along sides of the spacer.

In some implementations, a valve repair device for repairing a native valve of a patient comprises a spacer, a pair of anchors (e.g., paddles, latches, clamps, grippers, fasteners, etc.) configured to attach the valve repair device to the native valve of the patient, and at least one leak control extension extending from the spacer.

In some implementations, the pair of anchors are a pair of paddles coupled to the spacer. In some implementations, the pair of paddles are movable between an open position and a closed position.

In some implementations, the at least one leak control extension extends from the spacer. The leak control extension is configured to block retrograde blood flow along sides of the spacer.

In some implementations, the valve repair device further comprises a cap that is connected to the spacer. In some implementations, the at least one leak control extension is connected to the cap that is connected to the spacer. In some implementations, the at least one leak control extension is pivotally attached to the cap. In some implementations, the at least one leak control extension is connected directly to the spacer.

In some implementations, the valve repair device further comprises a pair of clasps, wherein the pair of anchors (e.g., a pair of paddles) and the pair of clasps are configured to attach the valve repair device to the native valve of the patient.

In some implementations, the spacer is configured to close a gap in the native valve of the patient when the valve repair device is attached to the native valve.

In some implementations, the at least one leak control extension comprises a deflector paddle having a flexible wire frame covered by a cloth barrier material. The deflector paddle can be connected to the spacer by one or more arms.

In some implementations, the flexible wire frame is configured to deform when positioned against a wall within the heart of the patient.

In some implementations, the at least one leak control extension comprises a pocket that includes a flexible wire frame that defines an opening of the pocket and a cloth barrier material that defines at least a portion of the interior of the pocket. In some implementations, the opening of the at least one leak control extension is configured to be positioned below one or more leaflets of the native valve when the valve repair device is attached to the native valve. In some implementations, the opening of the at least one leak control extension is configured to be positioned above a ventricular end of the one or more leaflets of the native valve when the valve repair device is attached to the native valve.

In some implementations, at least a portion of the leak control extension is configured to be positioned below a ventricular end of one or more leaflets of the native valve when the valve repair device is attached to the native valve.

In some implementations, the entire leak control extension is configured to be positioned above a ventricular end of one or more leaflets of the native valve when the valve repair device is attached to the native valve. In some implementations, the entire leak control extension is configured to be positioned below a ventricular end of one or more leaflets of the native valve when the valve repair device is attached to the native valve.

In some implementations, the at least one leak control extension comprises one or more deflector paddles and a barrier element. In some implementations, each of the one or more deflector paddles has a flexible wire frame covered by a cloth barrier material. In some implementations, the barrier element comprises at least one of a cloth material, a biocompatible material, bovine or porcine heart tissue, and a plastic membrane.

In some implementations, a valve repair device for repairing a native valve comprises at least one anchor (e.g., a paddle, latch, clamp, gripper, fastener, etc.) configured to attach the valve repair device to the native valve of the patient and at least one leak control extension extending from a portion of the valve repair device.

In some implementations, the at least one anchor is movable between an open position and a closed position.

In some implementations, the at least one leak control extension is configured to block retrograde blood flow adjacent or near the device.

In some implementations, the valve repair device further comprises a coaption element (e.g., coaptation element, spacer, etc.). The coaption element is configured to close a gap in the native valve of the patient when the valve repair device is attached to the native valve. In some implementations, the at least one leak control extension extends from the coaption element and is configured to block retrograde blood flow along sides of the coaption element.

In some implementations, the valve repair device further comprises a cap that is connected to the coaption element. In some implementations, the at least one leak control extension is connected directly to the coaption element. In some implementations, the at least one leak control extension is connected to the cap that is connected to the coaption element. In some implementations, the at least one leak control extension is pivotally attached to the cap.

In some implementations, the at least one anchor is coupled to the coaption element. In some implementations, the at least one anchor comprises a pair of paddles coupled to the coaption element.

In some implementations, the at least one anchor comprises a pair of paddles.

In some implementations, the valve repair device further comprises at least one clasp, wherein the at least one anchor and the at least one clasp are configured to attach the valve repair device to the native valve of the patient.

In some implementations, the at least one clasp is coupled to a cap of the device.

In some implementations, the at least one leak control extension comprises a deflector paddle having a flexible wire frame covered by a cloth barrier material. In some implementations, the deflector paddle is connected to the spacer by one or more arms. In some implementations, the flexible wire frame is configured to deform when positioned against a wall within the native valve of the patient.

In some implementations, the at least one leak control extension comprises a pocket that includes a flexible wire frame that defines an opening of the pocket and a cloth barrier material that defines at least a portion of the interior of the pocket. In some implementations, the opening of the at least one leak control extension is configured to be positioned below one or more leaflets of the native valve when the valve repair device is attached to the native valve. In some implementations, the opening of the at least one leak control extension is configured to be positioned above a ventricular end of the one or more leaflets of the native valve when the valve repair device is attached to the native valve.

In some implementations, at least a portion of the leak control extension is configured to be positioned below a ventricular end of one or more leaflets of the native valve when the valve repair device is attached to the native valve. In some implementations, the entire leak control extension is configured to be positioned above a ventricular end of one or more leaflets of the native valve when the valve repair device is attached to the native valve. In some implementations, the entire leak control extension is configured to be positioned below a ventricular end of one or more leaflets of the native valve when the valve repair device is attached to the native valve.

In some implementations, the at least one leak control extension comprises one or more deflector paddles and a barrier element. In some implementations, the one or more deflector paddles has a flexible wire frame covered by a cloth barrier material. In some implementations, the barrier element comprises at least one of a cloth material, a biocompatible material, bovine or porcine heart tissue, and a plastic membrane.

In some implementations, a valve repair system for repairing a native valve of a patient comprises a delivery sheath and a valve repair device that is deployable to a native valve of the patient by the delivery sheath.

In some implementations, the valve repair device comprises a coaption element or spacer, a pair of paddles (or other anchors) coupled to the spacer/coaption element, and at least one leak control extension extending from the spacer/coaption element, wherein the leak control extension is configured to block retrograde blood flow along sides of the spacer/coaption element.

In some implementations, the pair of paddles (or other anchors) are movable between an open position and a closed position, wherein the pair of paddles (or other anchors) are configured to attach the valve repair device to the native valve of the patient.

In some implementations, the system (e.g., the valve repair device of the system) further comprises a cap that is connected to the spacer/coaption element. In some implementations, the at least one leak control extension is connected to the cap that is connected to the spacer/coaption element. In some implementations, the at least one leak control extension is pivotally attached to the cap.

In some implementations, the at least one leak control extension is connected directly to the spacer/coaption element.

In some implementations, the system (e.g., the valve repair device of the system) further comprises pair of clasps, wherein the pair of paddles (or other anchors) and the pair of clasps are configured to attach the valve repair device to the native valve of the patient.

In some implementations, the spacer/coaption element is configured to close a gap in the native valve of the patient when the valve repair device is attached to the native valve.

In some implementations, the at least one leak control extension comprises a deflector paddle having a flexible wire frame covered by a cloth barrier material. In some implementations, the deflector paddle is connected to the spacer by one or more arms. In some implementations, the flexible wire frame is configured to deform when positioned against a wall within the heart of the patient.

In some implementations, the at least one leak control extension comprises a pocket that includes a flexible wire frame that defines an opening of the pocket and a cloth barrier material that defines at least a portion of the interior of the pocket. In some implementations, the opening of the at least one leak control extension is configured to be positioned below one or more leaflets of the native valve when the valve repair device is attached to the native valve. In some implementations, the opening of the at least one leak control extension is configured to be positioned above a ventricular end of the one or more leaflets of the native valve when the valve repair device is attached to the native valve.

In some implementations, at least a portion of the leak control extension is configured to be positioned below a ventricular end of one or more leaflets of the native valve when the valve repair device is attached to the native valve.

In some implementations, the entire leak control extension is configured to be positioned above a ventricular end of one or more leaflets of the native valve when the valve repair device is attached to the native valve. In some implementations, the entire leak control extension is configured to be positioned below a ventricular end of one or more leaflets of the native valve when the valve repair device is attached to the native valve.

In some implementations, the at least one leak control extension comprises one or more deflector paddles and a barrier element. In some implementations, each of the one or more deflector paddles has a flexible wire frame covered by a cloth barrier material. In some implementations, the barrier element comprises at least one of a cloth material, a biocompatible material, bovine or porcine heart tissue, and a plastic membrane.

In some implementations, a valve repair system for repairing a native valve of a patient comprises a delivery sheath and a valve repair device that is deployable to a native valve of the patient by the delivery sheath;

In some implementations, the valve repair device comprises at least one anchor (e.g., a paddle, latch, clamp, gripper, fastener, etc.) and at least one leak control extension extending from a portion of the device, wherein the leak control extension is configured to block retrograde blood flow adjacent or near the device.

In some implementations, the at least one anchor is movable between an open position and a closed position, wherein the pair of paddles are configured to attach the valve repair device to the native valve of the patient;

In some implementations, the system (e.g., the valve repair device of the system) further comprises a coaption element (e.g., spacer, coaptation element, etc.).

In some implementations, the at least one leak control extension extends from the coaption element and is configured to block retrograde blood flow along sides of the coaption element.

In some implementations, the system (e.g., the valve repair device of the system) further comprises a cap that is connected to the coaption element.

In some implementations, the at least one leak control extension is connected directly to the coaption element.

In some implementations, the at least one leak control extension is connected to the cap that is connected to the coaption element.

In some implementations, the at least one leak control extension is pivotally attached to the cap.

In some implementations, the at least one anchor is coupled to the coaption element. In some implementations, the at least one anchor comprises a pair of paddles coupled to the coaption element.

In some implementations, the at least one anchor comprises a pair of paddles.

In some implementations, the coaption element is configured to close a gap in the native valve of the patient when the valve repair device is attached to the native valve.

In some implementations, the system further comprises at least one pair clasp, wherein the at least one anchor and the at least one clasp are configured to attach the valve repair device to the native valve of the patient. In some implementations, the at least one clasp is coupled to a cap of the device.

In some implementations, the at least one leak control extension comprises a deflector paddle having a flexible wire frame covered by a cloth barrier material. In some implementations, the deflector paddle is connected to the coaption element by one or more arms. In some implementations, the flexible wire frame is configured to deform when positioned against a wall within the native valve of the patient.

In some implementations, the at least one leak control extension comprises a pocket that includes a flexible wire frame that defines an opening of the pocket and a cloth barrier material that defines at least a portion of the interior of the pocket.

In some implementations, the opening of the at least one leak control extension is configured to be positioned below one or more leaflets of the native valve when the valve repair device is attached to the native valve.

In some implementations, the opening of the at least one leak control extension is configured to be positioned above a ventricular end of the one or more leaflets of the native valve when the valve repair device is attached to the native valve.

In some implementations, at least a portion of the leak control extension is configured to be positioned below a ventricular end of one or more leaflets of the native valve when the valve repair device is attached to the native valve.

In some implementations, the entire leak control extension is configured to be positioned above a ventricular end of one or more leaflets of the native valve when the valve repair device is attached to the native valve. In some implementations, the entire leak control extension is configured to be positioned below a ventricular end of one or more leaflets of the native valve when the valve repair device is attached to the native valve.

In some implementations, the at least one leak control extension comprises one or more deflector paddles and a barrier element. In some implementations, each of the one or more deflector paddles has a flexible wire frame covered by a cloth barrier material. In some implementations, the barrier element comprises at least one of a cloth material, a biocompatible material, bovine or porcine heart tissue, and a plastic membrane.

In some implementations, a method of repairing a native valve of a patient comprises attaching a spacer or coaption element between leaflets of the native valve of the patient and blocking retrograde blood flow through gaps between the spacer and the leaflets.

In some implementations, the retrograde blood flow through the gaps is blocked without filling the gaps.

In some implementations, the retrograde blood flow through the gaps is blocked without filling any portion of the gaps.

In some implementations, the retrograde blood flow is blocked by extensions that are at least partially disposed on a ventricular side of the leaflets.

In some implementations, the retrograde blood flow is blocked by extensions that are completely disposed on a ventricular side of the leaflets.

In some implementations, the method further comprises positioning the spacer to deform one or more of the extensions against a wall within the heart of the patient.

The above method(s) can be performed on a living animal or on a simulation, such as on a cadaver, cadaver heart, simulator (e.g. with the body parts, heart, tissue, etc. being simulated), etc.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of embodiments of the present disclosure, a more particular description of example embodiments will be made by reference to various aspects of the appended drawings. It is appreciated that these drawings depict only typical embodiments of the present disclosure and are therefore not to be considered limiting of the scope of the disclosure. Moreover, while the figures can be drawn to scale for some embodiments, the figures are not necessarily drawn to scale for all embodiments. Embodiments and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a diastolic phase, in which the chordae tendineae are shown attaching the leaflets of the mitral and tricuspid valves to ventricle walls;

FIG. 4 illustrates a healthy mitral valve with the leaflets closed as viewed from an atrial side of the mitral valve;

FIG. 5 illustrates a dysfunctional mitral valve with a visible gap between the leaflets as viewed from an atrial side of the mitral valve;

FIG. 6 illustrates a mitral valve having a wide gap between the posterior leaflet and the anterior leaflet;

FIG. 7 illustrates a tricuspid valve viewed from an atrial side of the tricuspid valve;

FIGS. 8–14 show an example of an implantable prosthetic device, in various stages of deployment;

FIGS. 15–20 show the implantable prosthetic device of FIGS. 8–14 being delivered and implanted within a native valve;

FIG. 21 shows the implantable prosthetic device of FIGS. 8-14 implanted within a native valve;

FIG. 22 shows an implantable prosthetic device implanted in a first example position within a native valve;

FIG. 23 is a cross-sectional view of the implantable prosthetic device implanted in the first position within the native valve shown in FIG. 22, with the section taken along the plane indicated by line 23-23 in FIG. 22;

FIG. 24 shows the implantable prosthetic device implanted in the first position within the native valve shown in FIG. 22, viewed from the ventricle side of the native valve;

FIG. 25 shows the implantable prosthetic device implanted in the first position within the native valve shown in FIG. 22, viewed from the atrial side of the native valve;

FIG. 26 shows the implantable prosthetic device of FIG. 22 implanted in a second example position within the native valve, viewed from the ventricle side of the native valve;

FIG. 27 shows the implantable prosthetic device of FIG. 22 implanted in the second example position within the native valve shown in FIG. 26, viewed from the atrial side of the native valve;

FIG. 28 shows an example of an implantable prosthetic device implanted in a first example position within the native valve;

FIG. 29 is a cross-sectional view of the implantable prosthetic device implanted in the first position within the native valve shown in FIG. 28, with the section taken along the plane indicated by line 29-29 in FIG. 28;

FIG. 30 shows the implantable prosthetic device implanted in the first position within the valve shown in FIG. 28, viewed when the heart is in a diastolic phase;

FIG. 31 is a cross-sectional view of the implantable prosthetic device implanted in the first position within the native valve shown in FIG. 30, with the section taken along the plane indicated by line 31-31 shown in FIG. 30;

FIG. 32 shows the implantable prosthetic device implanted in the first position within the valve shown in FIG. 28, viewed when the heart is in a systolic phase;

FIG. 33 is a cross-sectional view of the implantable prosthetic device implanted in the first position within the native valve shown in FIG. 32, with the section taken along the plane indicated by line 33-33 shown in FIG. 32;

FIG. 34 shows the implantable prosthetic device implanted in the first position within the native valve shown in FIG. 28, viewed from a ventricle side of the native valve;

FIG. 35 shows the implantable prosthetic device implanted in the first position within the native valve shown in FIG. 28, viewed from an atrial side of the native valve;

FIG. 36 shows the implantable prosthetic device of FIG. 28 implanted in a second example position within the native valve, viewed from the ventricle side of the native valve;

FIG. 37 shows the implantable prosthetic device of FIG. 28 implanted in the second example position within the native valve shown in FIG. 36, viewed from the atrial side of the native valve;

FIG. 38A shows an example of an implantable prosthetic device implanted in a first example position within the native valve;

FIG. 38B shows an example of an implantable prosthetic device implanted in a first example position within the native valve;

FIG. 39A is a cross-sectional view of the implantable prosthetic device implanted in the first position within the native valve shown in FIG. 38A, with the section taken along the plane indicated by line 39A-39A in FIG. 38A;

FIG. 39B is a cross-sectional view of the implantable prosthetic device implanted in the first position within the native valve shown in FIG. 38B, with the section taken along the plane indicated by line 39B-39B in FIG. 38B;

FIG. 40A shows the implantable prosthetic device implanted in the first position within the native valve shown in FIG. 38A, viewed when the heart is in a diastolic phase;

FIG. 40B shows the implantable prosthetic device implanted in the first position within the native valve shown in FIG. 38B, viewed when the heart is in a diastolic phase;

FIG. 41A is a cross-sectional view of the implantable prosthetic device implanted in the first position within the native valve shown in FIG. 40A, with the section taken along the plane indicated by line 41A-41A shown in FIG. 40A;

FIG. 41B is a cross-sectional view of the implantable prosthetic device implanted in the first position within the native valve shown in FIG. 40B, with the section taken along the plane indicated by line 41B-41B shown in FIG. 40B;

FIG. 42A shows the implantable prosthetic device implanted in the first position within the native valve shown in FIG. 38A, viewed when the heart is in a systolic phase;

FIG. 42B shows the implantable prosthetic device implanted in the first position within the native valve shown in FIG. 38B, viewed when the heart is in a systolic phase;

FIG. 43A is a cross-sectional view of the implantable prosthetic device implanted in the first position within the native valve shown in FIG. 42A, with the section taken along the plane indicated by line 43A-43A shown in FIG. 42A;

FIG. 43B is a cross-sectional view of the implantable prosthetic device implanted in the first position within the native valve shown in FIG. 42B, with the section taken along the plane indicated by line 43B-43B shown in FIG. 42B;

FIG. 44A shows the implantable prosthetic device implanted in the first position within the native valve shown in FIG. 38A, viewed from a ventricle side of the native valve;

FIG. 44B shows the implantable prosthetic device implanted in the first position within the native valve shown in FIG. 38B, viewed from a ventricle side of the native valve;

FIG. 45A shows the implantable prosthetic device implanted in the first position within the native valve shown in FIG. 38A, viewed from an atrial side of the native valve;

FIG. 45B shows the implantable prosthetic device implanted in the first position within the native valve shown in FIG. 38B, viewed from an atrial side of the native valve;

FIG. 46A shows the implantable prosthetic device of FIG. 38A implanted in a second example position within the native valve, viewed from the ventricle side of the native valve;

FIG. 46B shows the implantable prosthetic device of FIG. 38B implanted in a second example position within the native valve, viewed from the ventricle side of the native valve;

FIG. 47A shows the implantable prosthetic device of FIG. 38A implanted in the second example position within the mitral valve shown in FIG. 46A, viewed from the atrial side of the native valve;

FIG. 47B shows the implantable prosthetic device of FIG. 38B implanted in the second example position within the mitral valve shown in FIG. 46B, viewed from the atrial side of the native valve;

FIG. 48A shows an example of an implantable prosthetic device implanted in a first example position within the native valve;

FIG. 48B shows an example of an implantable prosthetic device implanted in a first example position within the native valve;

FIG. 49A is a cross-sectional view of the implantable prosthetic device implanted in the first position within the native valve shown in FIG. 48A, with the section taken along the plane indicated by line 49A-49A in FIG. 48A;

FIG. 49B is a cross-sectional view of the implantable prosthetic device implanted in the first position within the native valve shown in FIG. 48B, with the section taken along the plane indicated by line 49B-49B in FIG. 48B;

FIG. 50A shows the implantable prosthetic device implanted in the first position within the native valve shown in FIG. 48A, viewed when the heart is in a diastolic phase;

FIG. 50B shows the implantable prosthetic device implanted in the first position within the native valve shown in FIG. 48B, viewed when the heart is in a diastolic phase;

FIG. 51A is a cross-sectional view of the implantable prosthetic device implanted in the first position within the native valve shown in FIG. 50A, with the section taken along the plane indicated by line 51A-51A shown in FIG. 50A;

FIG. 51B is a cross-sectional view of the implantable prosthetic device implanted in the first position within the native valve shown in FIG. 50B, with the section taken along the plane indicated by line 51B-51B shown in FIG. 50B;

FIG. 52A shows the implantable prosthetic device implanted in the first position within the native valve shown in FIG. 48A, viewed when the heart is in a systolic phase;

FIG. 52B shows the implantable prosthetic device implanted in the first position within the native valve shown in FIG. 48B, viewed when the heart is in a systolic phase;

FIG. 53A is a cross-sectional view of the implantable prosthetic device implanted in the first position within the native valve shown in FIG. 52A, with the section taken along the plane indicated by line 53A-53A shown in FIG. 52A;

FIG. 53B is a cross-sectional view of the implantable prosthetic device implanted in the first position within the native valve shown in FIG. 52B, with the section taken along the plane indicated by line 53B-53B shown in FIG. 52B;

FIG. 54A shows the implantable prosthetic device implanted in the first position within the native valve shown in FIG. 48A, viewed from a ventricle side of the native valve;

FIG. 54B shows the implantable prosthetic device implanted in the first position within the native valve shown in FIG. 48B, viewed from a ventricle side of the native valve;

FIG. 55A shows the implantable prosthetic device implanted in the first position within the native valve shown in FIG. 48A, viewed from an atrial side of the native valve;

FIG. 55B shows the implantable prosthetic device implanted in the first position within the native valve shown in FIG. 48B, viewed from an atrial side of the native valve;

FIG. 56A shows the implantable prosthetic device of FIG. 48A implanted in a second example position within the native valve, viewed from the ventricle side of the native valve;

FIG. 56B shows the implantable prosthetic device of FIG. 48B implanted in a second example position within the native valve, viewed from the ventricle side of the native valve;

FIG. 57A shows the implantable prosthetic device of FIG. 48A implanted in the second example position within the native valve shown in FIG. 56A, viewed from the atrial side of the native valve;

FIG. 57B shows the implantable prosthetic device of FIG. 48B implanted in the second example position within the native valve shown in FIG. 56B, viewed from the atrial side of the native valve;

FIG. 58 shows an example of an implantable prosthetic device implanted in a first example position within the native valve;

FIG. 59 is a cross-sectional view of the implantable prosthetic device implanted in the first position within the native valve shown in FIG. 58, with the section taken along the plane indicated by line 59-59 in FIG. 58;

FIG. 60 shows the implantable prosthetic device implanted in the first position within the native valve shown in FIG. 58, viewed when the heart is in a diastolic phase;

FIG. 61 is a cross-sectional view of the implantable prosthetic device implanted in the first position within the native valve shown in FIG. 60, with the section taken along the plane indicated by line 61-61 shown in FIG. 60;

FIG. 62 shows the implantable prosthetic device implanted in the first position within the native valve shown in FIG. 58, viewed when the heart is in a systolic phase;

FIG. 63 is a cross-sectional view of the implantable prosthetic device implanted in the first position within the native valve shown in FIG. 62, viewed along the line 63-63 shown in FIG. 62;

FIG. 64 shows the implantable prosthetic device implanted in the first position within the native valve shown in FIG. 58, viewed from a ventricle side of the native valve;

FIG. 65 shows the implantable prosthetic device implanted in the first position within the native valve shown in FIG. 58, viewed from an atrial side of the native valve;

FIG. 66 shows the implantable prosthetic device of FIG. 58 implanted in a second example position within the native valve, viewed from the ventricle side of the native valve;

FIG. 67 shows the implantable prosthetic device of FIG. 58 implanted in the second example position within the native valve shown in FIG. 66, viewed from the atrial side of the native valve;

FIG. 68 shows an example of an implantable prosthetic device implanted in a first example position within the native valve;

FIG. 69 is a cross-sectional view of the implantable prosthetic device implanted in the first position within the native valve shown in FIG. 68, with the section taken along the plane indicated by line 69-69 in FIG. 68;

FIG. 70 shows the implantable prosthetic device implanted in the first position within the valve shown in FIG. 68, viewed when the heart is in a diastolic phase;

FIG. 71 is a cross-sectional view of the implantable prosthetic device implanted in the first position within the native valve shown in FIG. 70, with the section taken along the plane indicated by line 71-71 shown in FIG. 70;

FIG. 72 shows the implantable prosthetic device implanted in the first position within the valve shown in FIG. 68, viewed when the heart is in a systolic phase;

FIG. 73 is a cross-sectional view of the implantable prosthetic device implanted in the first position within the native valve shown in FIG. 72, with the section taken along the plane indicated by line 73-73 shown in FIG. 72;

FIG. 74 shows the implantable prosthetic device implanted in the first position within the native valve shown in FIG. 68, viewed from a ventricle side of the native valve;

FIG. 75 shows the implantable prosthetic device implanted in the first position within the native valve shown in FIG. 68, viewed from an atrial side of the native valve;

FIG. 76 shows the implantable prosthetic device of FIG. 68 implanted in a second example position within the native valve, viewed from the ventricle side of the native valve;

FIG. 77 shows the implantable prosthetic device of FIG. 68 implanted in the second example position within the native valve shown in FIG. 76, viewed from the atrial side of the native valve;

FIG. 78 is a bottom view of a more specific example of the implantable prosthetic device shown in FIGS. 38A-47A;

FIG. 79 is a bottom view of a more specific example of the implantable prosthetic device shown in FIGS. 38A-47A;

FIG. 80 is a front view of a more specific example of the implantable prosthetic device shown in FIGS. 48A-57A;

FIG. 81 is a bottom view of the implantable prosthetic device shown in FIG. 80;

FIG. 82 shows an example of an implantable prosthetic device implanted in a first example position within the native valve;

FIG. 83 is a cross-sectional view of the implantable prosthetic device implanted in the first position within the native valve shown in FIG. 82, with the section taken along the plane indicated by line 83-83 in FIG. 82;

FIG. 84 shows the implantable prosthetic device implanted in the first position within the native valve shown in FIG. 82, viewed when the heart is in a diastolic phase;

FIG. 85 is a cross-sectional view of the implantable prosthetic device implanted in the first position within the native valve shown in FIG. 84, with the section taken along the plane indicated by line 85-85 shown in FIG. 84;

FIG. 86 shows the implantable prosthetic device implanted in the first position within the native valve shown in FIG. 82, viewed when the heart is in a systolic phase;

FIG. 87 is a cross-sectional view of the implantable prosthetic device implanted in the first position within the native valve shown in FIG. 86, viewed along the line 87-87 shown in FIG. 86;

FIG. 88 shows the implantable prosthetic device implanted in the first position within the native valve shown in FIG. 82, viewed from a ventricle side of the native valve;

FIG. 89 shows the implantable prosthetic device implanted in the first position within the native valve shown in FIG. 82, viewed from an atrial side of the native valve;

FIG. 90 shows the implantable prosthetic device of FIG. 82 implanted in a second example position within the native valve, viewed from the ventricle side of the native valve; and

FIG. 91 shows the implantable prosthetic device of FIG. 82 implanted in the second example position within the native valve shown in FIG. 90, viewed from the atrial side of the native valve.

### DETAILED DESCRIPTION

The following description refers to the accompanying drawings, which illustrate specific embodiments of the present disclosure. Other embodiments having different structures and operation do not depart from the scope of the present disclosure.

Example implementations of the present disclosure are directed to systems, devices, methods, etc. for repairing a defective heart valve. It should be noted that various embodiments of native valve repair devices, systems for delivery of native valve repair devices, and systems for removal of implanted native valve repair devices are disclosed herein, and any combination of these options can be made unless specifically excluded. In other words, individual components of the disclosed devices and systems can be combined unless mutually exclusive or otherwise physically impossible. Further, the techniques and methods can be performed on a living animal or on a simulation, such as on a cadaver, cadaver heart, simulator (e.g. with the body parts, heart, tissue, etc. being simulated), etc.

As described herein, when one or more components are described as being connected, joined, affixed, coupled, attached, or otherwise interconnected, such interconnection may be direct as between the components or may be indirect such as through the use of one or more intermediary components. Also, as described herein, reference to a "member," “component,” or “portion” shall not be limited to a single structural member, component, or element but can include an assembly of components, members, or elements. Also, as described herein, the terms “substantially” and “about” are defined as at least close to (and includes) a given value or state (preferably within 10% of, more preferably within 1% of, and most preferably within 0.1% of).

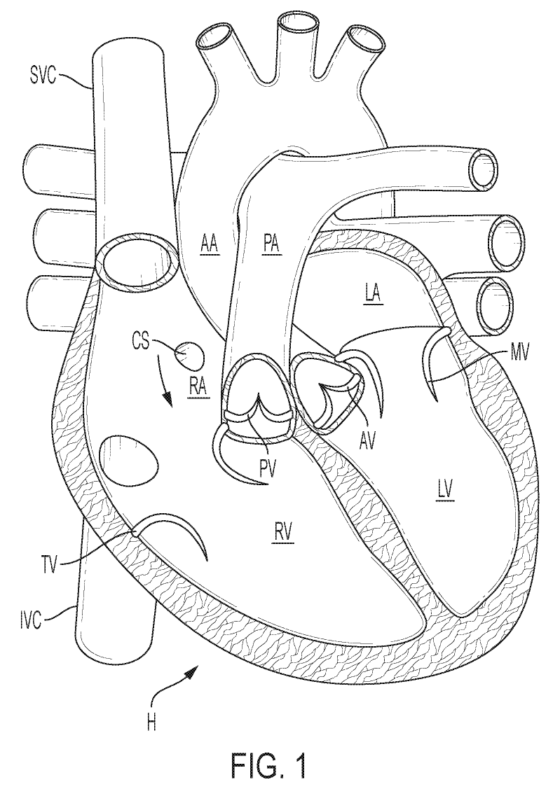

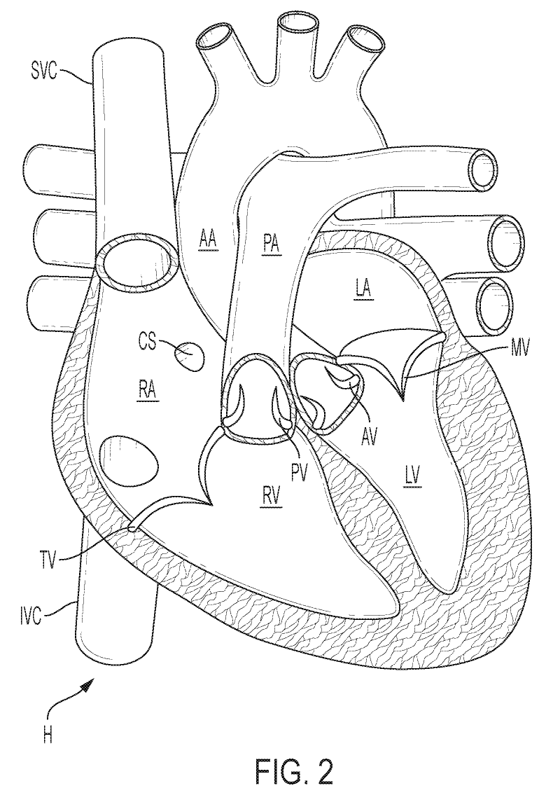

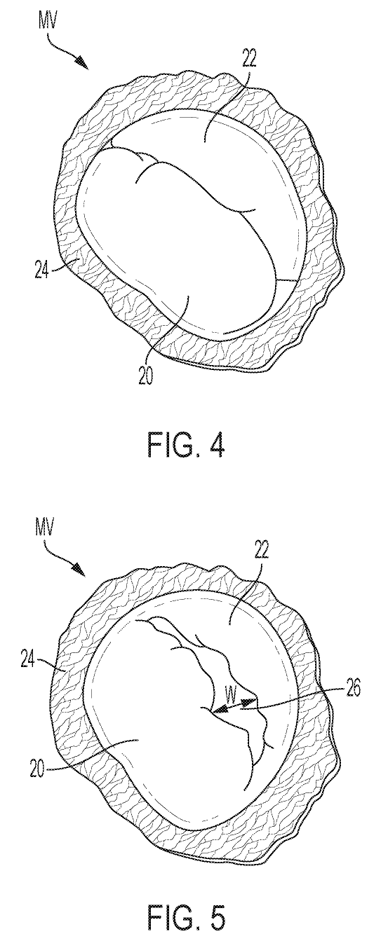

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 4 and 5) extending inward across the respective orifices that come together or "coapt" in the flow stream to form the one-way, fluid-occluding surfaces. The native valve repair systems of the present application are described primarily with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA, and blood is collected in the left atrium from the pulmonary vein. In one example implementation, the devices described by the present application are used to repair the function of a defective mitral valve MV. That is, the devices are configured to help close the leaflets of the mitral valve to prevent blood from regurgitating from the left ventricle LV and back into the left atrium LA.

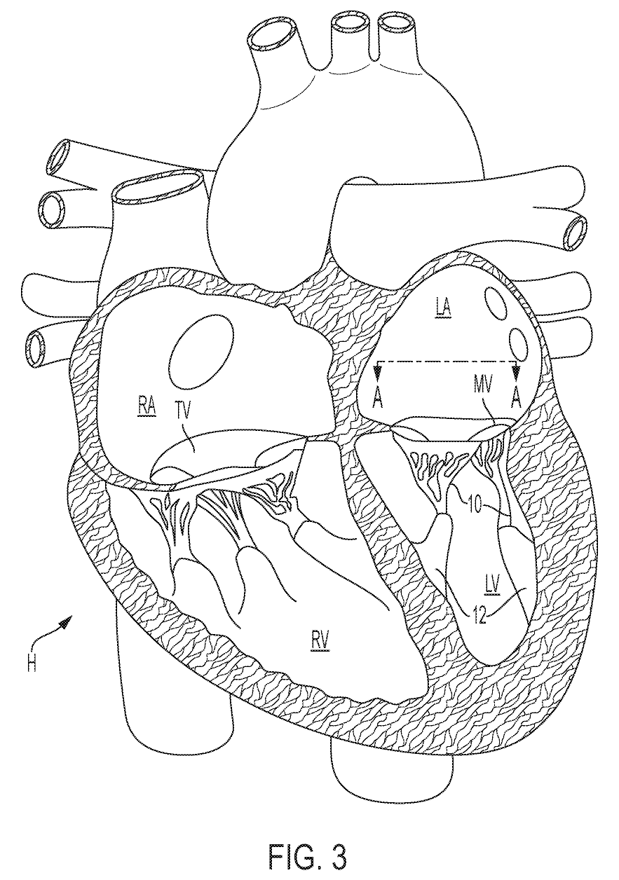

Referring now to FIGS. 1–7, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIG. 3, the mitral valve MV is anchored to the wall of the left ventricle LV by chordae tendineae 10. The chordae tendineae 10 are cord-like tendons that connect the papillary muscles 12 (i.e., the muscles located at the base of the chordae tendineae and within the walls of the left ventricle) to the leaflets 20, 22 of the mitral valve MV. The papillary muscles 12 serve to limit the movements of the mitral valve MV and prevent the mitral valve from being reverted. The mitral valve MV opens and closes in response to pressure changes in the left atrium LA and the left ventricle LV. The papillary muscles do not open or close the mitral valve MV. Rather, the papillary muscles brace the mitral valve MV against the high pressure needed to circulate blood throughout the body. Together the papillary muscles and the chordae tendineae are known as the subvalvular apparatus, which functions to keep the mitral valve MV from prolapsing into the left atrium LA when the mitral valve closes.

Various disease processes can impair proper function of one or more of the native valves of the heart H. These disease processes include degenerative processes (e.g., Barlow’s Disease, fibroelastic deficiency), inflammatory processes (e.g., Rheumatic Heart Disease), and infectious processes (e.g., endocarditis). In addition, damage to the left ventricle LV or the right ventricle RV from prior heart attacks (i.e., myocardial infarction secondary to coronary artery disease) or other heart diseases (e.g., cardiomyopathy) can distort a native valve’s geometry, which can cause the native valve to dysfunction. However, the vast majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in two different ways: (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow.

The second type of valve malfunction, valve regurgitation, occurs when the leaflets of the valve do not close completely thereby causing blood to leak back into the prior chamber (e.g., causing blood to leak from the left ventricle to the left atrium). There are three mechanisms by which a native valve becomes regurgitant—or incompetent—which include Carpentier’s type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier’s type II malfunction involves prolapse of one or more leaflets of a native valve above a plane of coaptation. A Carpentier’s type III malfunction involves restriction of the motion of one or more leaflets of a native valve such that the leaflets are abnormally constrained below the plane of the annulus. Leaflet restriction can be caused by rheumatic disease (Ma) or dilation of a ventricle (IIIb).

Referring to FIG. 4, when a healthy mitral valve MV is in a closed position, the anterior leaflet 20 and the posterior leaflet 22 coapt, which prevents blood from leaking from the left ventricle LV to the left atrium LA. Referring to FIG. 5, regurgitation occurs when the anterior leaflet 20 and/or the posterior leaflet 22 of the mitral valve MV is displaced into the left atrium LA during systole. This failure to coapt causes a gap 26 between the anterior leaflet 20 and the posterior leaflet 22, which allows blood to flow back into the left atrium LA from the left ventricle LV during systole. As set forth above, there are several different ways that a leaflet (e.g. leaflets 20, 22 of mitral valve MV) may malfunction, which can thereby lead to regurgitation.

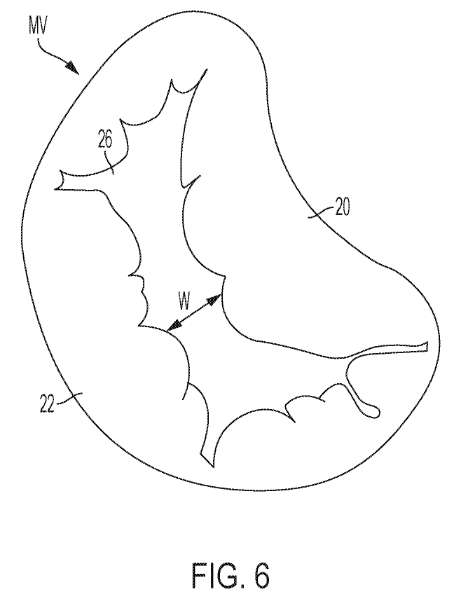

Referring to FIG. 6, in certain situations, the mitral valve MV of a patient can have a wide gap 26 between the anterior leaflet 20 and the posterior leaflet 22 when the mitral valve is in a closed position (i.e., during the systolic phase). For example, the gap 26 can have a width W between about 2.5 mm and about 17.5 mm, such as between about 5 mm and about 15 mm, such as between about 7.5 mm and about 12.5 mm, such as about 10 mm. In some situations, the gap 26 can have a width W greater than 15 mm. In any of the above-mentioned situations, a valve repair device is desired that is capable of engaging the anterior leaflet 20 and the posterior leaflet 22 to close the gap 26 and prevent regurgitation of blood through the mitral valve MV.

Although stenosis or regurgitation can affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) is primarily responsible for circulating the flow of blood throughout the body, malfunction of the mitral valve MV or the aortic valve AV is particularly problematic and often life threatening. Accordingly, because of the substantially higher pressures on the left side of the heart, dysfunction of the mitral valve MV or the aortic valve AV is much more problematic.

Malfunctioning native heart valves may either be repaired or replaced. Repair typically involves the preservation and correction of the patient’s native valve. Replacement typically involves replacing the patient’s native valve with a biological or mechanical substitute. Typically, the aortic valve AV and pulmonary valve PV are more prone to stenosis. Because stenotic damage sustained by the leaflets is irreversible, the most conventional treatments for a stenotic aortic valve or stenotic pulmonary valve are removal and replacement of the valve with a surgically implanted heart valve, or displacement of the valve with a transcatheter heart valve. The mitral valve MV and the tricuspid valve TV are more prone to deformation of leaflets, which, as described above, prevents the mitral valve or tricuspid valve from closing properly and allows for regurgitation or back flow of blood from the ventricle into the atrium (e.g., a deformed mitral valve MV may allow for regurgitation or back flow from the left ventricle LV to the left atrium LA). The regurgitation or back flow of blood from the ventricle to the atrium results in valvular insufficiency. Deformations in the structure or shape of the mitral valve MV or the tricuspid valve TV can be repairable. In addition, regurgitation can occur due to the chordae tendineae 10 becoming dysfunctional (e.g., the chordae tendineae may stretch or rupture), which allows the anterior leaflet 20 and the posterior leaflet 22 to be reverted such that blood is regurgitated into the left atrium LA. The problems occurring due to dysfunctional chordae tendineae can be repaired by repairing the chordae tendineae or the structure of the mitral valve (e.g., by securing the leaflets 20, 22 at the affected portion of the mitral valve).

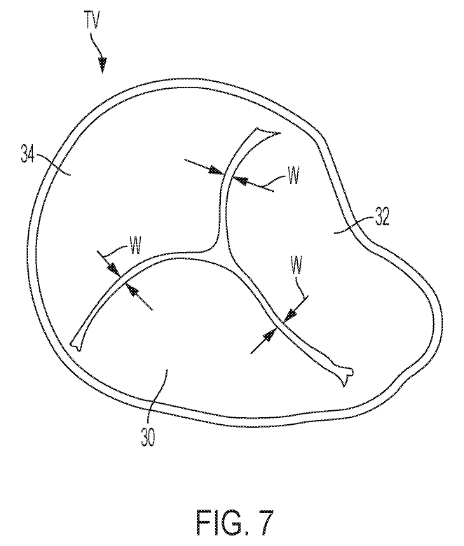

The devices and procedures disclosed herein make reference to repairing the structure of a mitral valve or removing an implanted repair device from the mitral valve. However, it should be understood that the devices and concepts provided herein can be used to repair any native valve or any component of a native valve. Referring now to FIG. 7, any of the devices and concepts provided herein can be used to repair the tricuspid valve TV. For example, the devices and concepts provided herein can be used between any two of the anterior leaflet 30, septal leaflet 32, and posterior leaflet 34 to prevent regurgitation of blood from the right ventricle into the right atrium. In addition, any of the devices and concepts provided herein can be used on all three of the leaflets 30, 32, 34 together to prevent regurgitation of blood from the right ventricle to the right atrium. That is, the valve repair devices provided herein can be centrally located between the three leaflets 30, 32, 34.

The concepts disclosed in the present patent application can be applied to a variety of different valve repair devices. Some examples of valve repair devices that the concepts disclosed herein can be applied to are disclosed in US Provisional Patent Application Serial No. 62/744,031, filed on October 10, 2018, Patent Cooperation Treaty Application No. PCT/US2019/012707, filed on January 8, 2019, and Patent Cooperation Treaty No. PCT/US2018/028189 which are incorporated herein by reference in their entireties.

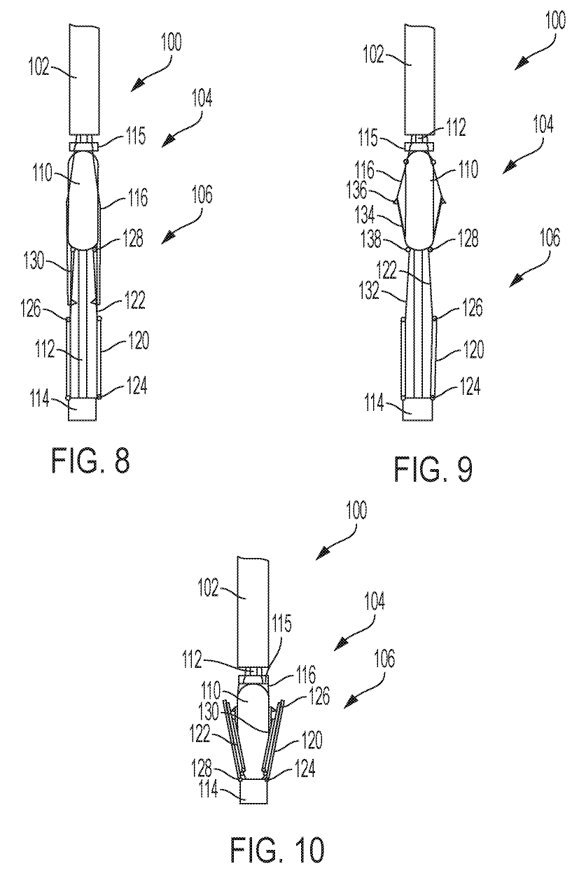

FIGS. 8-14 illustrate an example of a valve repair device. An example implantable prosthetic device can have a coaptation or coaption element (e.g., a spacer, etc.) and at least one anchor. The coaption element is configured to be positioned within the native heart valve orifice to help fill the space and form a more effective seal, thereby reducing or preventing regurgitation described above. The coaption element can have a structure that is impervious to blood and that allows the native leaflets to close around the coaption element during ventricular systole to block blood from flowing from the left or right ventricle back into the left or right atrium, respectively. The prosthetic device can be configured to seal against two or three native valve leaflets; that is, the device may be used in the native mitral (bicuspid) and tricuspid valves. The coaption element is sometimes referred to herein as a spacer because the coaption element can fill a space between improperly functioning native mitral or tricuspid leaflets that do not close completely.

The coaption element (e.g., spacer, etc.) can have various shapes. In some embodiments, the coaption element can have an elongated cylindrical shape having a round cross-sectional shape. In some embodiments, the coaption element can have an oval cross-sectional shape, a crescent cross-sectional shape, or various other non-cylindrical shapes. The coaption element can have an atrial portion positioned in or adjacent to the left atrium, a ventricular or lower portion positioned in or adjacent to the left ventricle, and a side surface that extends between the native mitral leaflets. In embodiments configured for use in the tricuspid valve, the atrial or upper portion is positioned in or adjacent to the right atrium, and the ventricular or lower portion is positioned in or adjacent to the right ventricle, and the side surface that extends between the native tricuspid leaflets.

The anchor can be configured to secure the device to one or both of the native mitral leaflets such that the coaption element is positioned between the two native leaflets. In embodiments configured for use in the tricuspid valve, the anchor is configured to secure the device to one, two, or three of the tricuspid leaflets such that the coaption element is positioned between the three native leaflets. In some embodiments, the anchor can attach to the coaption element at a location adjacent the ventricular portion of the coaption element. In some embodiments, the anchor can attach to a shaft or actuation wire or other actuation element, to which the coaption element is also attached. In some embodiments, the anchor and the coaption element can be positioned independently. In some embodiments, the anchor and the coaption element can be positioned simultaneously. The anchor can be configured to grasp the leaflets.

The prosthetic device can be configured to be implanted via a delivery sheath. Additional information regarding examples of delivery methods can be found in U.S. Pat. No. 8,449,599 and U.S. Patent Application Publication Nos. 2014/0222136, 2014/0067052, and 2016/0331523, each of which is incorporated herein by reference in its entirety. Further, these methods can be performed on a living animal or on a simulation, such as on a cadaver, cadaver heart, simulator (e.g. with the body parts, heart, tissue, etc. being simulated), etc. mutatis mutandis.

Referring now to FIGS. 8–14, an example of an implantable prosthetic device 100 schematically illustrated is shown in various stages of deployment. However, the implantable prosthetic device can take a wide variety of different forms as mentioned above. For example, the features of the present application can be included with any of the implantable prosthetic devices disclosed in US Provisional Patent Application Serial No. 62/744,031, Patent Cooperation Treaty Application No. PCT/US2019/012707, and/or Patent Cooperation Treaty No. PCT/US2018/028189. The device 100 can include any other features for an implantable prosthetic device discussed in the present application, and the device 100 can be positioned to engage valve tissue (e.g., leaflets 20, 22) as part of any suitable valve repair system (e.g., any valve repair system disclosed in the present application).

The device 100 can be deployed from a delivery sheath 102 and can include a coaption portion 104 and/or an anchor portion 106. The coaption portion 104 of the device 100 includes a coaption element or spacer 110 that is adapted to be implanted between the leaflets of the native valve (e.g., native mitral valve, native tricuspid valve, etc.) and is slidably attached to an actuation member or actuation element 112 (e.g., a wire, shaft, rod, line, suture, tether, etc.). The anchor portion 106 is actuatable between open and closed conditions and can take a wide variety of forms, such as, for example, paddles, latches, clasps, fasteners, gripping elements, or the like. Actuation of the actuation element 112 (e.g., actuation of an actuation wire, etc.) opens and closes the anchor portion 106 of the device 100 to grasp the mitral valve leaflets during implantation. The actuation element 112 can take a wide variety of different forms. For example, the actuation element can be threaded such that rotation of the actuation element moves the anchor portion 106 relative to the coaption portion 104. Or, the actuation element may be unthreaded, such that pushing or pulling the actuation element 112 moves the anchor portion 106 relative to the coaption portion 104.

In some implementations, the anchor portion 106 of the device 100 includes outer paddles 120 and inner paddles 122 that are connected between a cap 114 and the coaption element 110 by portions 124, 126, 128. The portions 124, 126, 128 can be jointed, hinged, and/or flexible to move between all of the positions described below. The interconnection of the outer paddles 120, the inner paddles 122, the coaption element 110, and the cap 114 by the portions 124, 126, and 128 can constrain the device to the positions and movements illustrated herein. In some implementations, the device includes only one outer paddle 120 and one inner paddle 122, and these can be configured in different ways.

The actuation member or actuation element 112 extends through the delivery sheath and/or a pusher tube/rod and/or the coaption element or spacer 110 to the cap 114 at the distal connection of the anchor portion 106. Extending and retracting the actuation element 112 increases and decreases the spacing between the coaption element 110 and the cap 114, respectively. An optional attaching means or collar (not shown) removably attaches the coaption element 110 to a pusher tube or rod and/or delivery sheath 102 so that the actuation element 112 slides along the actuation element 112 during actuation to open and close the paddles 120, 122 of the anchor portion 106. After the device 100 is connected to valve tissue, if the device 100 needs to be removed from the valve tissue, a retrieval device can be used to connect to the collar 115 such that the actuation element can extend through the collar 115 and the coaption element 110 to engage the anchor portion 106 to open the paddles 120, 122 and remove the device 100 from the valve tissue. Examples of retrieval devices that could be used are shown in PCT Application No. PCT/US2019/062391 filed Nov. 20, 2019, which is incorporated herein by reference in its entirety.

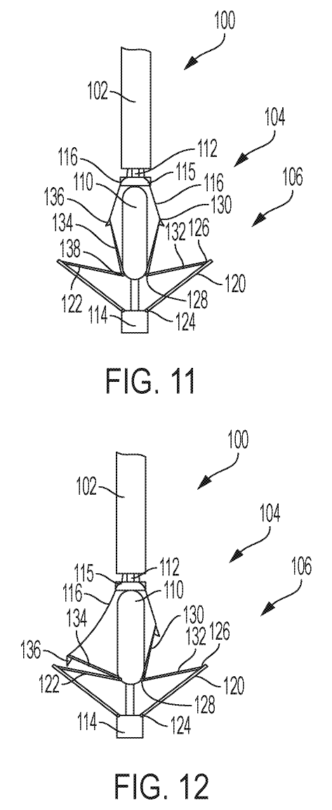

Referring now to FIG. 11, the anchor portion 106 includes attachment portions or gripping members. The illustrated gripping members are shown as barbed clasps 130 that include a base or fixed arm 132, a moveable arm 134, barbs 136, and a flex, hinge, or joint portion 138. Although, other friction-enhancing elements can be substituted for the barbs. The fixed arms 132 are attached to the inner paddles 122, with the flex, hinge, or joint portion 138 disposed proximate the coaption element 110. The barbed clasps have flat surfaces and do not fit in a recess of the paddle. Rather, the flat portions of the barbed clasps 130 are disposed against the surface of the inner paddle 122. The flex, hinge, or joint portion 138 provides a spring force between the fixed and moveable arms 132, 134 of the barbed clasp 130. The joint portion 138 can be any suitable flexible portion, hinge, or joint, such as a flexible joint or hinge, a spring joint or hinge, a pivot joint or hinge, or the like. In some embodiments, the flex, hinge, or joint portion 138 is a flexible piece of material integrally formed with the fixed and moveable arms 132, 134. The fixed arms 132 are attached to the inner paddles 122 and remain stationary relative to the inner paddles 122 when the moveable arms 134 are opened to open the barbed clasps 130 and expose the barbs 136. The barbed clasps 130 are opened by applying tension to actuation lines 116 attached to the moveable arms 134, thereby causing the moveable arms 134 to move, flex, and/or pivot on the flex, hinge, or joint portions 138.

During implantation, the paddles 120, 122 are opened and closed to capture or grasp the native mitral valve leaflets between the paddles 120, 122 and the coaption element 110. The barbed clasps 130 further secure the native leaflets by engaging the leaflets with barbs 136 and pinching the leaflets between the moveable and fixed arms 134, 132. The barbs 136 of the barbed clasps 130 increase friction with the leaflets or may partially or completely puncture the leaflets. The actuation lines 116 can be actuated independently or separately so that each barbed clasp 130 can be opened and closed independently or separately. Separate/independent operation allows one leaflet to be grasped at a time, or for the repositioning of a clasp 130 on a leaflet that was insufficiently grasped, without altering a successful grasp on the other leaflet. The barbed clasps 130 not only open and close independent from each other but can fully be opened and closed independent from the position of the inner paddle 122, thereby allowing leaflets to be captured in a variety of positions as the particular situation requires.

The barbed clasps 130 can be opened independently or separately by pulling on an attached actuation line 116 (or other actuation means) that extends through the delivery sheath 102 to the barbed clasp 130. The actuation line 116 can take a wide variety of forms, such as, for example, a line, a suture, a wire, a rod, a catheter, or the like. The barbed clasps 130 can be spring loaded or otherwise biased so that in the closed position the barbed clasps 130 continue to provide a pinching force on the captured or grasped native leaflet. This pinching force can remain constant or positive regardless of the position of the inner paddles 122. Barbs 136 of the barbed clasps 130 can pierce the native leaflets to further secure the native leaflets.

Referring now to FIG. 8, the device 100 is shown in an elongated or fully open condition for deployment from the delivery sheath 102. The device 100 is loaded in the delivery sheath 102 in the fully open position, because the fully open position takes up the least space and allows the smallest catheter to be used (or the largest implantable device 100 to be used for a given catheter size). In the elongated condition the cap 114 is spaced apart from the coaption element 110 such that the paddles 120, 122 of the anchor portion 106 are fully open or fully extended. In some embodiments, an angle formed between the interior of the outer and inner paddles 120, 122 is approximately 180 degrees. The barbed clasps 130 are kept in a closed condition during deployment through the delivery sheath 102 so that the barbs 136 (FIG. 11) do not catch or damage the sheath or tissue in the patient’s heart.

Referring now to FIG. 9, the device 100 is shown in an elongated detangling condition, similar to FIG. 8, but with the barbed clasps 130 in a fully open position, ranging from about 140 degrees to about 200 degrees, about 170 degrees to about 190 degrees, or about 180 degrees between fixed and moveable portions of the barbed clasps 130. Fully opening the paddles 120, 122 and the clasps 130 has been found to improve ease of detanglement from anatomy of the patient during implantation of the device 100.

Referring now to FIG. 10, the device 100 is shown in a shortened or fully closed condition. The compact size of the device 100 in the shortened condition allows for easier maneuvering and placement within the heart. To move the device 100 from the elongated condition to the shortened condition, the actuation element 112 is retracted to pull the cap 114 towards the coaption element 110. The joints, hinges, or flexible connections 126 between the outer paddle 120 and inner paddle 122 are limited or constrained in movement such that compression forces acting on the outer paddle 120 from the cap 114 being retracted towards the coaption element 110 cause the paddles 120, 122 or gripping elements to move radially outward. During movement from the open to closed position, the outer paddles 120 maintain an acute angle with the actuation element 112. The outer paddles 120 can optionally be biased toward a closed position. The inner paddles 122 during the same motion move through a considerably larger angle as they are oriented away from the coaption element 110 in the open condition and collapse along the sides of the coaption element 110 in the closed condition. In some embodiments, the inner paddles 122 are thinner and/or narrower than the outer paddles 120, and the joint, hinge, or flexible portions 126, 128 connected to the inner paddles 122 can be thinner and/or more flexible. For example, this increased flexibility can allow more movement than the joint, hinge, or flexible portion 124 connecting the outer paddle 124 to the cap 114. In some embodiments, the outer paddles 120 are narrower than the inner paddles 122. The joint or flexible portions 126, 128 connected to the inner paddles 122 can be more flexible, for example, to allow more movement than the joint or flexible portion 124 connecting the outer paddle 124 to the cap 114. In some embodiments, the inner paddles 122 can be the same width or substantially the same width as the outer paddles.

Referring now to FIGS. 11–13, the device 100 is shown in a partially open, capture-ready or grasp-ready condition. To transition from the fully closed to the partially open condition, the actuation element 112 (e.g., and actuation wire, actuation shaft, etc.) is extended to push the cap 114 away from the coaption element 110, thereby pulling on the outer paddles 120, which in turn pull on the inner paddles 122, causing the anchor portion 106 to partially unfold. The actuation lines 116 are also retracted to open the clasps 130 so that the leaflets can be captured or grasped. In some embodiments, like the example illustrated by FIG. 11, the pair of inner and outer paddles 122, 120 are moved in unison, rather than independently, by a single actuation element 112. Also, the positions of the clasps 130 may be dependent on the positions of the paddles 122, 120. For example, referring to FIG. 10 closing the paddles 122, 120 can also close the clasps. In some embodiments, the paddles 120, 122 can be independently controllable. For example, the device 100 can have two actuation elements and two independent caps, such that one independent wire and cap are used to control one paddle, and the other independent wire and cap are used to control the other paddle.

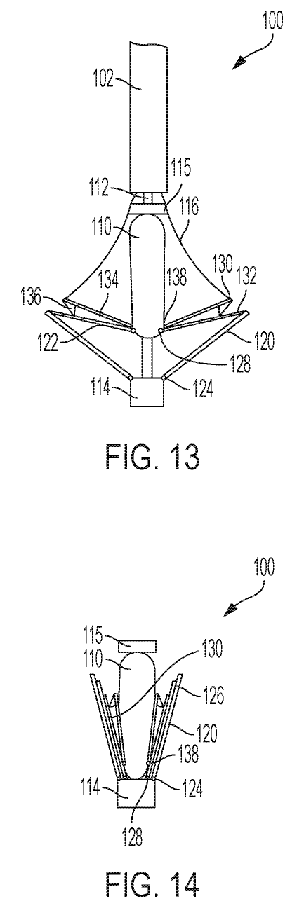

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 may be repeatedly actuated to repeatedly open and close the barbed clasps 130.

Referring now to FIG. 14, the device 100 is shown in a fully closed and deployed condition. The delivery sheath 102 and actuation element 112 are retracted, and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the jointed, hinged, or flexible portions 124, 126, 128, 138, and/or the inner and outer paddles 122, and/or an additional biasing component can be formed of metals such as steel or shape-memory alloy, such as Nitinol—produced in a wire, sheet, tubing, or laser sintered powder—and can biased to hold the outer paddles 120 closed around the coaption element or spacer 110 and the barbed clasps 130 pinched around native leaflets. In some embodiments, the paddles 120, 122 can be configured to open and close with the beating of the heart and corresponding opening and closing of the native valve.

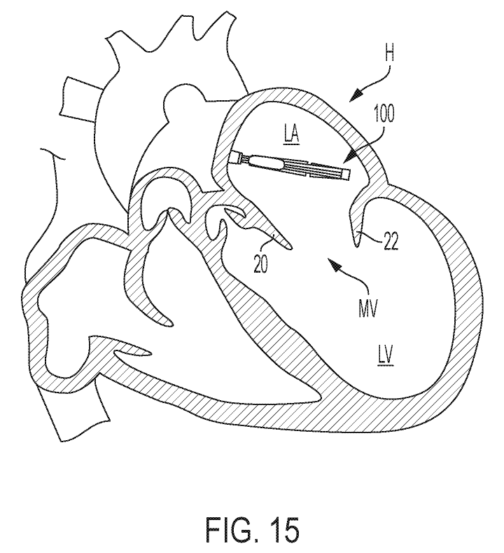

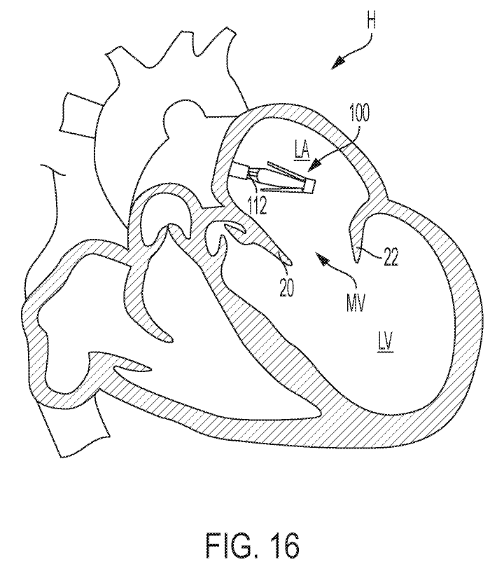

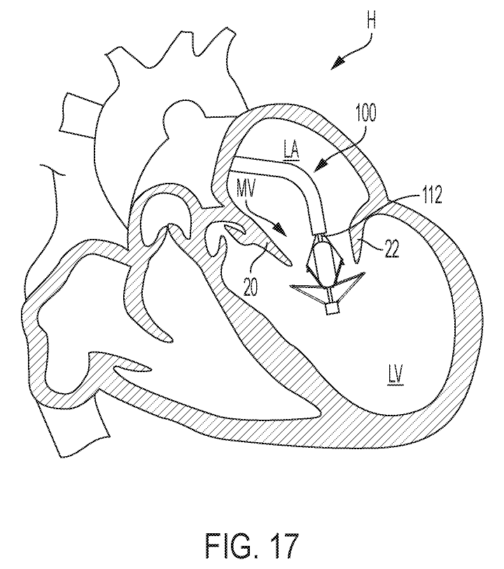

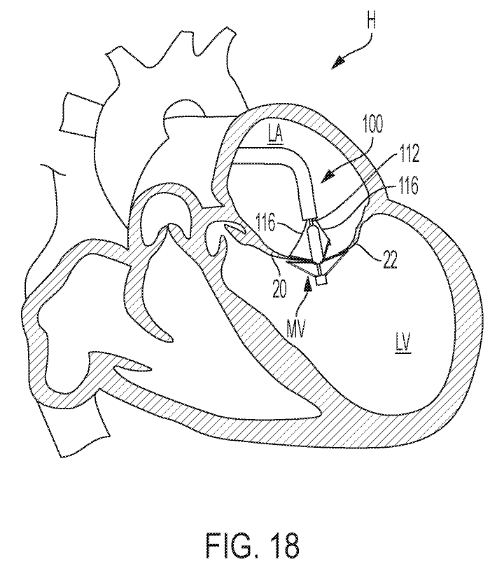

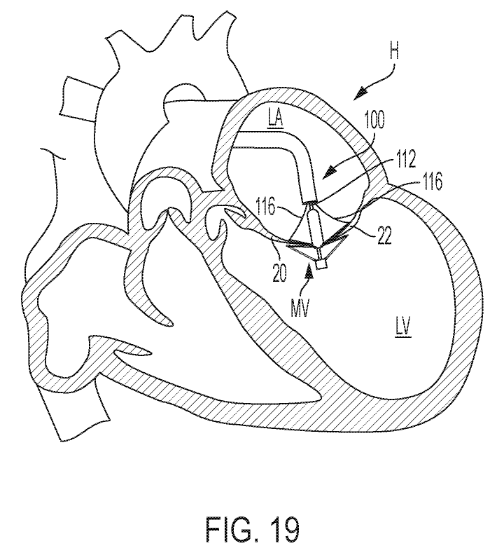

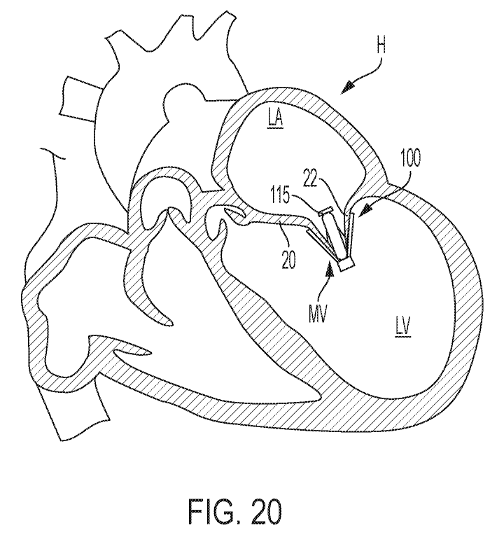

Referring now to FIGS. 15–20, the implantable device 100 of FIGS. 8–14 is shown being delivered and implanted within the native mitral valve MV of the heart H. Referring now to FIG. 15, the delivery sheath is inserted into the left atrium LA through the septum and the device 100 is deployed from the delivery sheath in the fully open condition. The actuation element 112 is then retracted to move the device 100 into the fully closed condition shown in FIG. 16. As can be seen in FIG. 17, the device 100 is moved into position within the mitral valve MV into the ventricle LV and partially opened so that the leaflets 20, 22 can be captured or grasped. Referring now to FIG. 18, an actuation line 116 is extended to close one of the clasps 130, capturing a leaflet 20. FIG. 19 shows the other actuation line 116 being then extended to close the other clasp 130, capturing the remaining leaflet 22. Lastly, as can be seen in FIG. 20, the delivery sheath 102 and actuation element 112 and actuation lines 116 are then retracted and the device 100 is fully closed and deployed in the native mitral valve MV.

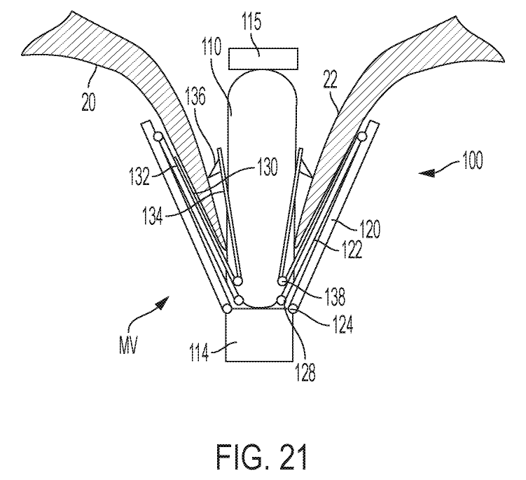

Referring now to FIG. 21, the device 100 of FIGS. 8-14 is shown implanted within a native valve, e.g., native mitral valve MV, in the fully closed position. The implanted device 100 has outer paddles 120, inner paddles 122, clasps 130, a coaption element 110 (e.g., a spacer, etc.), and a cap 114. The outer paddles 120 and the inner paddles 122 are connected between the cap 114 and the coaption element 110 by portions 124, 126, 128 (which can be jointed and/or flexible to move between various positions). The coaption element 110 is adapted to be implanted between the leaflets 20, 22 of the native valve. The clasps 130 are configured to connect device 100 to the leaflets 20, 22. In some embodiments, the clasps 130 include a fixed arm that is attached to the inner paddle 122 and a movable arm 134 that has a friction-enhancing element (e.g., barb, ridges, rough surface, adhesive, etc.) for engaging the leaflets 20, 22 of the native valve. In some implementations, the device 100 has only one outer paddle 120, only one inner paddle 122, and only one clasp (e.g., a barbed clasp, etc.), and these can be configured in different ways.

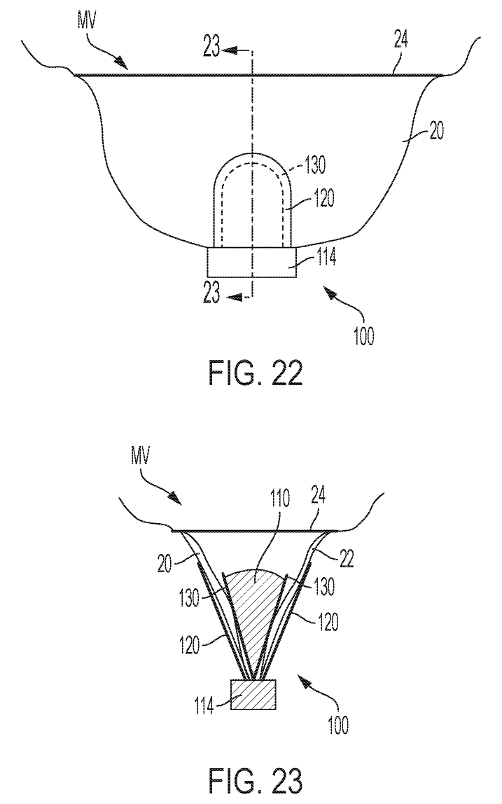

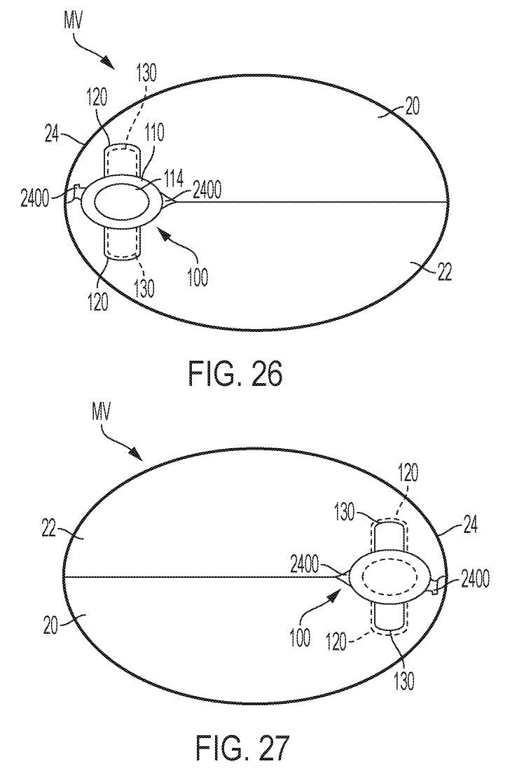

Referring to FIGS. 22-27, an implantable prosthetic device 100 is attached to the leaflets 20, 22 of a native valve (illustrated, for example, as mitral valve MV, but could be similarly used on another valve such as the tricuspid valve) in the fully closed position. In the embodiment shown in FIGS. 22-25, the implantable prosthetic device 100 is attached to the native valve at a substantially central location within the annulus 24. It should be understood, however, that the implantable prosthetic device 100 can be attached to the leaflets 20, 22 at any location within the native valve (e.g., the location shown in FIGS. 26-27). The device 100 is shown as having a pair of paddles 120, a pair of clasps 130, a coaption element 110, and a cap 114. However, the implantable prosthetic device 100 can take any suitable form, such as, for example, any formed described in the present application.

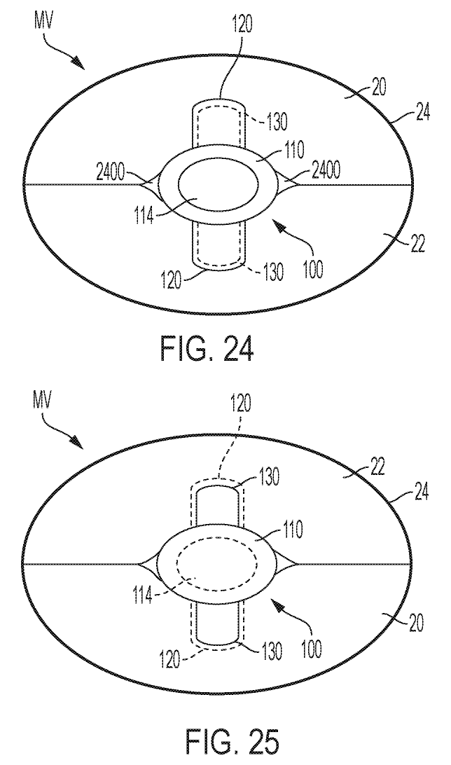

Referring to FIGS. 24 and 25, during the systolic phase, the leaflets 20, 22 coapt around the implantable prosthetic device 100 to prevent regurgitation of blood from the left ventricle to the left atrium. In certain situations, however, the leaflets 20, 22 may not completely coapt around the device 100, which may cause one or more openings or jets 2400 to form between the device 100 and one or more of the leaflets 20, 22. The openings 2400 may form at the location where both leaflets 20, 22 and the device 100 meet. The openings 2400 can also form, however, at any point where the device 100 and a single leaflet 20, 22 meet. These openings 2400 allow for blood to regurgitate into the left atrium during the systolic phase.

Referring to FIGS. 26 and 27, the device 100 can be placed within the native valve or mitral valve MV in a location near the annulus 24. Similar to the device 100 being located in a more central location within the mitral valve MV (as shown in FIGS. 24 and 25), openings 2400 may form where both leaflets 20, 22 and the device 100 meet. As shown in the illustrated embodiment, when the device 100 is placed near the annulus 24 the force on the mitral valve MV by the device 100 may cause the portions of the leaflets adjacent to the annulus 24 to bunch up or pucker and may cause an opening 2400 have a deformed shape. For example, when the device 100 is placed near the annulus, it is more difficult to align the leaflets in the device. When the leaflets are offset from one another in the device 100, the portions of the leaflets adjacent to the annulus 24 may bunch up or pucker and may cause an opening 2400 with a deformed shape on one or both sides of the device.

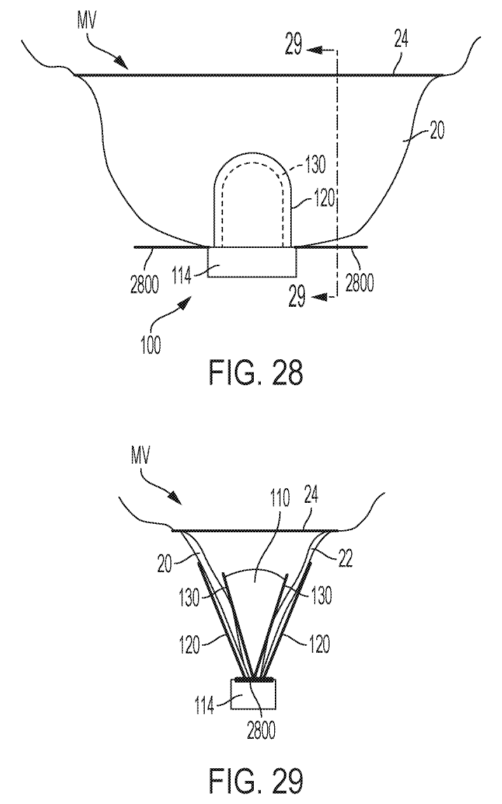

FIGS. 28-91 show various embodiments of an implantable prosthetic device 100 that includes one or more leak control mechanisms or leak control extensions 2800, where the leak control extensions 2800 allow blood to flow past or over the leak control extensions 2800 when the heart is in the diastolic phase (i.e. blood flow from the atrium to the ventricle) and that blocks or deflects at least a portion of blood flow that would otherwise regurgitate through the openings 2400 when the heart is in the systolic phase (i.e. blood flow from the ventricle to the atrium). The leak control extension 2800 can take a wide variety of different forms. For example, the leak control extension 2800 can have the configuration of a substantially flat deflecting paddle, a curved deflecting paddle, a bag or sack that opens toward the cap, a deflecting paddle and cloth assembly, and the like. The leak control extensions 2800 can be made from a wide variety of different materials. For example, the leak control extensions can be made from thin plastic, a wire frame with a cloth covering, cloth without a frame, cloth with a plastic frame, any combination thereof, etc. The leak control extensions 2800 can be positioned in a wide variety of different ways. For example, the leak control extensions can be positioned at the bottom of the native valve leaflets, below the bottom of the native valve leaflets, between the bottom of the native valve leaflets and the native valve annulus, or such that a portion of the leak control extension is below the native valve leaflets and a portion of the leak control extension is between the bottom of the native valve leaflets and the native valve annulus.

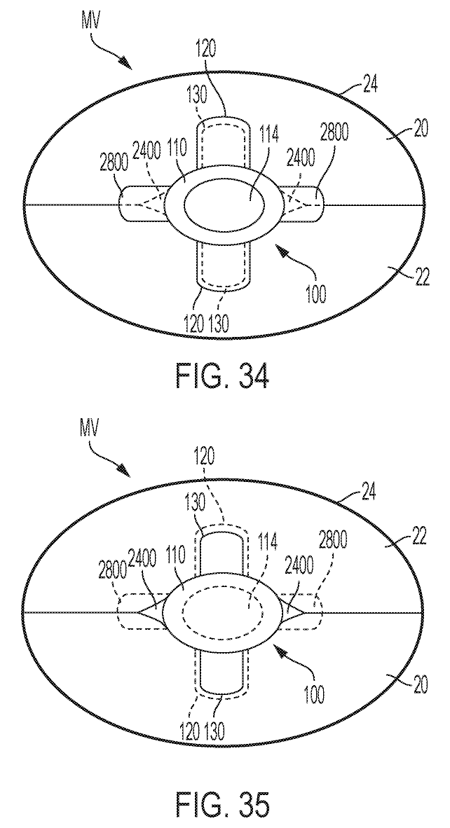

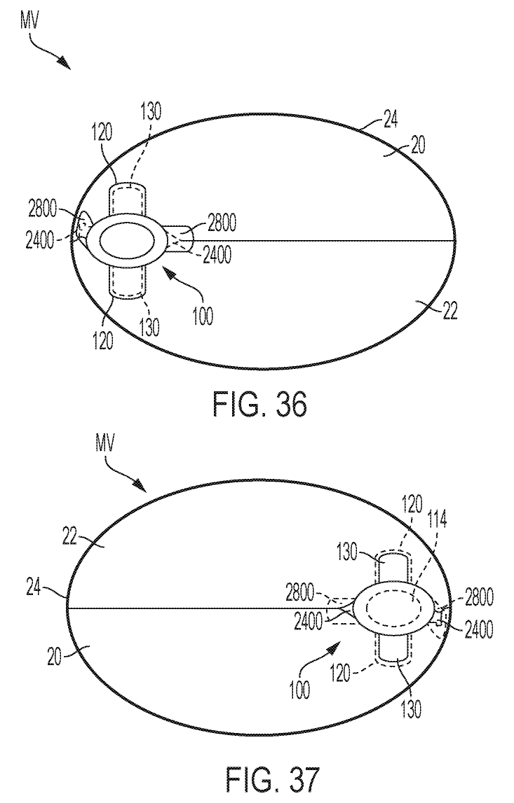

FIGS. 28-37 show an example of an implantable prosthetic device 100 attached to the leaflets 20, 22 of a native valve (illustrated, for example, as mitral valve MV, but could be similarly used on another valve such as the tricuspid valve) in the fully closed position, where the device 100 includes one or more leak control extensions 2800. In the embodiment shown in FIGS. 28-35, the implantable prosthetic device 100 is attached to the native valve at a substantially central location relative to the annulus 24. It should be understood, however, that the implantable prosthetic device 100 can be attached to the leaflets 20, 22 at any location within the native valve (e.g., the location shown in FIGS. 36-37). The device 100 has a pair of paddles 120, a pair of clasps 130, a coaption element 110 (e.g., a spacer, etc.), a cap 114, and leak control extensions 2800. However, the implantable prosthetic device 100 can take any suitable form, such as, for example, any form described in the present application, any of the implantable prosthetic devices disclosed in US Provisional Patent Application Serial No. 62/744,031, Patent Cooperation Treaty Application No. PCT/US2019/012707, and/or Patent Cooperation Treaty No. PCT/US2018/028189 in combination with the leak control extensions 2800.

In the example illustrated by FIGS. 28 and 29, the leak control extensions 2800 include deflecting paddles 2801 connected to the top surface of the cap 14 and positioned below the native valve leaflets. The leak control extension(s) 2800 are configured to block blood from the ventricle from reaching the opening 2400 during the systolic phase. That is, the leak control extension(s) 2800 extend from the device (e.g., from the cap 114 of the device 100) such that the leak control extensions are positioned to prevent blood from moving through any jets or openings between the leaflets 20, 22 of the native valve during the systolic phase. In the illustrated embodiment, the leak control extensions 2800 are positioned in the left ventricle LV when the device 100 is attached to the leaflets 20, 22. In the illustrated embodiment, the device 100 has two leak control extensions 2800 that are attached to a top surface of the cap 14. While the illustrated embodiment is shown as having two leak control extensions 2800, it should be understood that the device 100 can have any suitable number of leak control extensions.

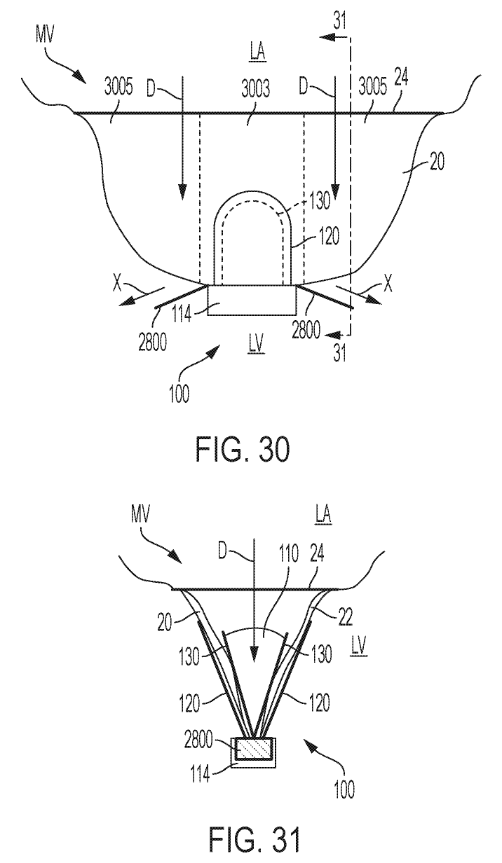

Referring to FIGS. 30 and 31, during the diastolic phase, the leaflets 20, 22 of the mitral valve MV open and blood moves from the left atrium LA to the left ventricle LV. As the device 100 is connected to the leaflets 20, 22, a portion 3003 of the mitral valve MV is substantially blocked by the device 100 when the leaflets 20, 22 are in the open position, while other portion(s) 3005 of the mitral valve are open such that blood can move into the left ventricle LV. The blood moves through the open portions 3005 in the direction D and engages the deflecting paddles 2801 of the leak control extensions 2800. In some embodiments, the blood provides a force on the deflecting paddles 2801 that causes the deflecting paddles 2801 to pivot about the cap 114 or flex with respect to the cap 114 such that the blood moves around the leak control extensions 2800 in the direction X.

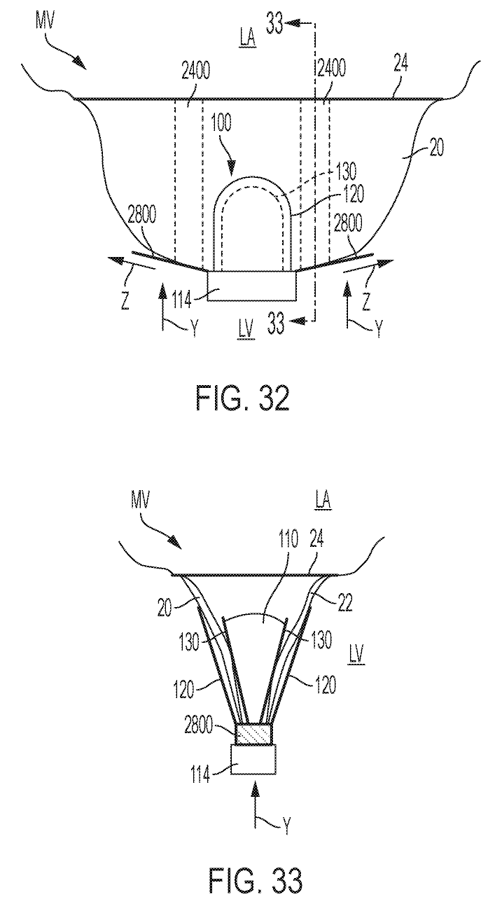

Referring to FIGS. 32-35, during the systolic phase, the leaflets 20, 22 of the mitral valve MV coapt to prevent blood from regurgitating into the left atrium LA as blood is pushed from the left ventricle and into the aorta, and the device 100 is connected to the leaflets 20, 22 to help facilitate the coapting or coaptation of the leaflets. In certain situations, however, the leaflets 20, 22 may not completely coapt around the device 100, which may cause one or more openings or jets 2400 to form between the device 100 and one or more of the leaflets 20, 22. The openings 2400 may form at the location where both leaflets 20, 22 and the device 100 meet. The openings 2400 can also form, however, at any point where the device 100 and a single leaflet 20, 22 meet. The leak control extensions 2800 are positioned to prevent blood from regurgitating into the left atrium through the one or more openings 2400.

Referring to FIGS. 32 and 33, contraction of the left ventricle LV pushes blood toward the mitral valve MV in the direction Y and engages the deflecting paddles 2801 of the leak control extensions 2800, which prevents blood from moving through the openings 2400 and into the left atrium LA. In some embodiments, the blood provides a force on the deflecting paddles 2801 that causes the deflecting paddles 2801 to pivot about the cap 114 or flex relative to the cap, such that the blood is directed away from the openings 2400 around the leak control extensions 2800 in the direction Z.

Referring to FIGS. 36 and 37, the device 100 shown in FIGS. 28-35 can be placed within the native valve in a location near the annulus 24. Similar to the device 100 being located in a more central location within the native valve (as shown in FIGS. 28-35), openings 2400 may form where both leaflets 20, 22 and the device 100 meet. As shown in the illustrated embodiment, when the device 100 is placed near the annulus 24, the force on the native valve by the device 100 may cause the opening 2400 adjacent to the annulus 24 to have a deformed shape. In some embodiments, the leak control extensions 2800 are flexible such that forces applied to the leak control extensions cause the leak control extensions to compress. For example, as shown in FIGS. 36 and 37, the leak control extension 2800 adjacent to the annulus 24 is pressed against the annulus 24 (or the side wall of the ventricle), which causes the leak control extension 2800 to compress into a deformed shape. This compression of the leak control extensions 2800 allows the leak control extensions to cover the deformed opening 2400 that is adjacent to the annulus 24. The flexible leak control extensions 2800 are advantageous because it allows the device 100 to be positioned within the native valve at various locations without causing irritation to the annulus 24 or the side walls of the ventricle or the atrium. In addition, the flexible leak control extensions 2800 are advantageous because they are capable of taking the form of any deformed openings caused by the connection between the device 100 and the leaflets 20, 22.

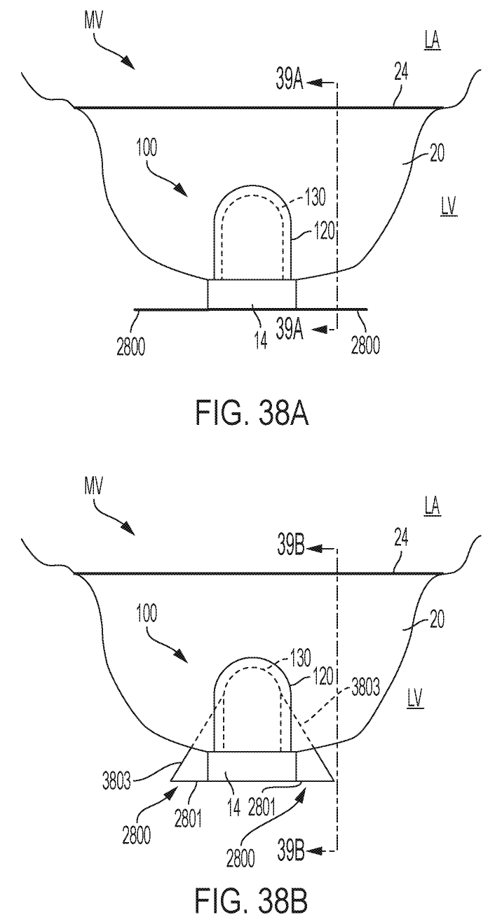

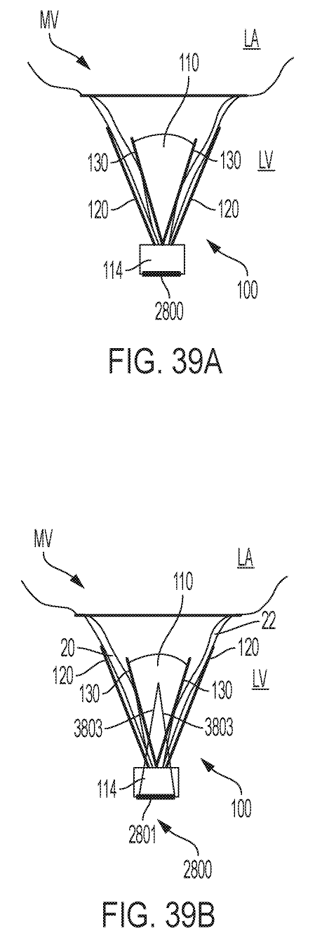

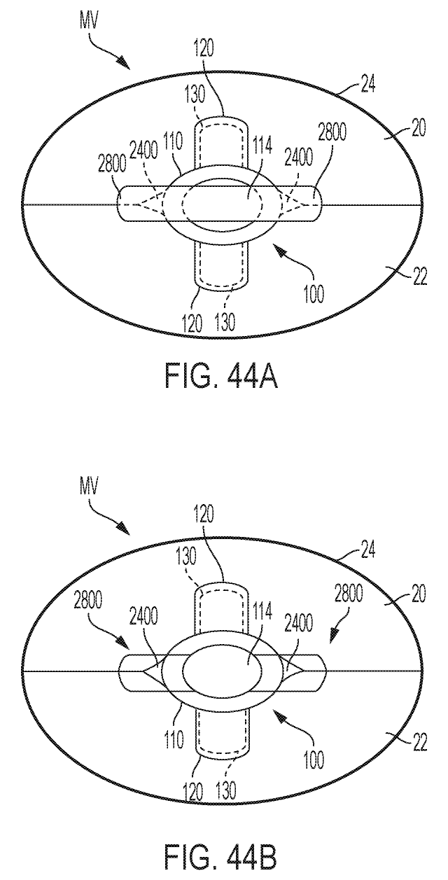

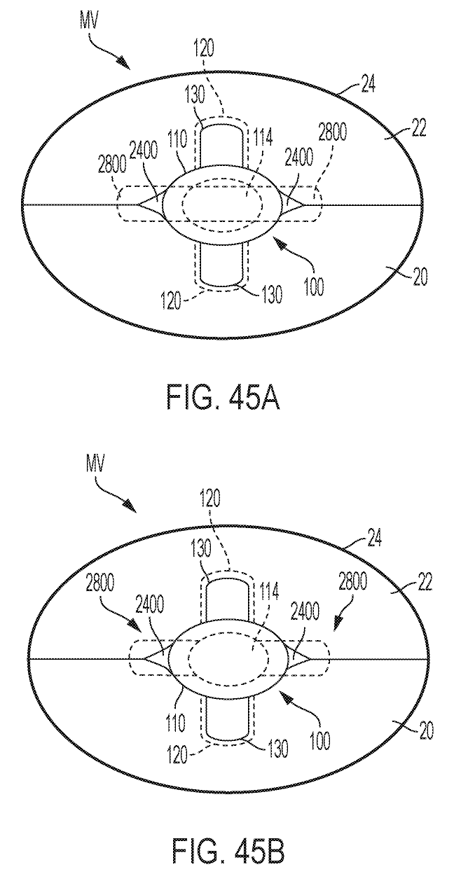

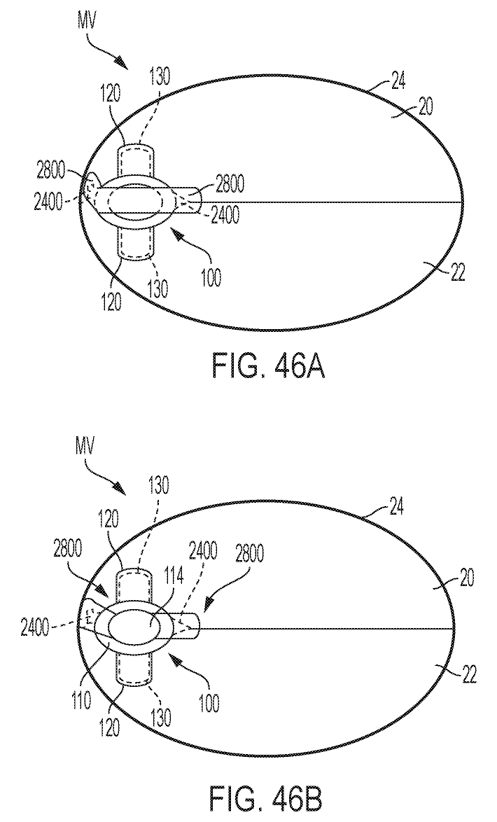

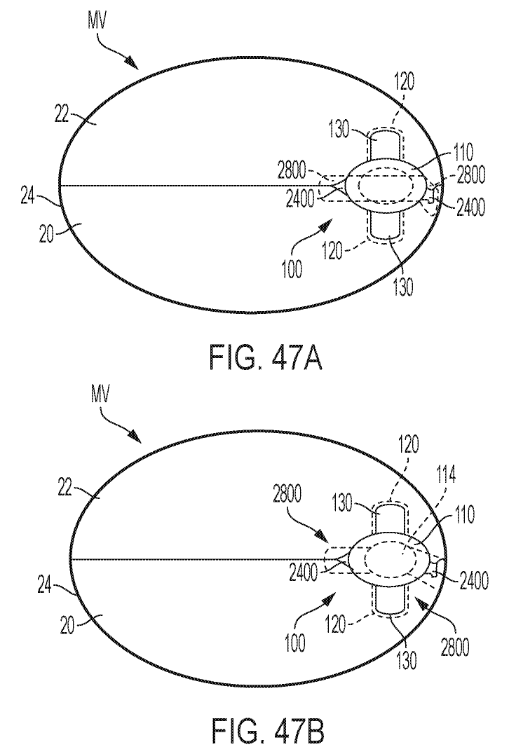

FIGS. 38A-47A show an example of an implantable prosthetic device 100 attached to leaflets 20, 22 of a native valve (illustrated, for example, as mitral valve MV, but could be similarly used on another valve such as the tricuspid valve) in the fully closed position, where the device 100 includes one or more leak control extensions 2800. In the embodiment shown in FIGS. 38A-45A, the implantable prosthetic device 100 is attached to the native valve at a substantially central location within the annulus 24. It should be understood, however, that the implantable prosthetic device 100 can be attached to the leaflets 20, 22 at any location within the native valve (e.g., the location shown in FIGS. 46A-47A). The device 100 has a pair of paddles 120, a pair of clasps 130, a coaption element 110 (e.g., a spacer etc.), a cap 114, and at least one leak control extension 2800. However, the implantable prosthetic device 100 can take any suitable form, such as, for example, any formed described in the present application in combination with at least one leak control extension 2800.

The leak control extension(s) 2800 are configured to prevent blood from regurgitating from the ventricle and into the atrium during the systolic phase. That is, the leak control extension(s) 2800 extend from the device (e.g., from the end of the cap 114 of the device 100) such that the leak control extensions are positioned to prevent blood from moving through any jets or openings between the leaflets 20, 22 of the mitral valve MV during the systolic phase. In the illustrated embodiment, the leak control extensions 2800 include deflecting paddles 2801 that are positioned in the left ventricle LV when the device 100 is attached to the leaflets 20, 22. In the illustrated embodiment, the device 100 has two leak control extensions 2800 that are attached to a bottom surface of the cap 114. The leak control extensions 2800 can be connected to each other (as shown in the illustrated embodiment), or the leak control extensions 2800 can be separate extensions. While the illustrated embodiment is shown as having two leak control extensions 2800, it should be understood that the device 100 can have any suitable number of leak control extensions.

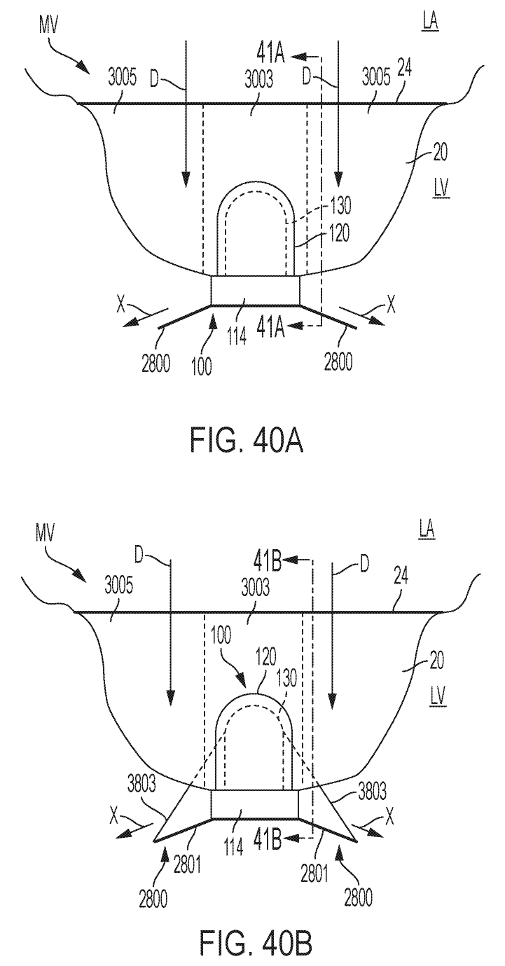

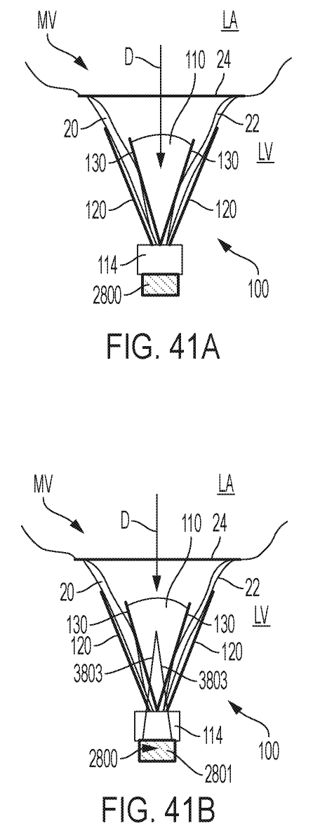

Referring to FIGS. 40A and 41A, during the diastolic phase, the leaflets 20, 22 of the mitral valve MV open and blood moves from the left atrium LA to the left ventricle LV. As the device 100 is connected to the leaflets 20, 22, a portion 3003 of the mitral valve MV is substantially blocked by the device 100 when the leaflets 20, 22 are in the open position, while other portion(s) 3005 of the mitral valve are open such that blood can move into the left ventricle LV. The blood moves through the open portions 3005 in the direction D and engages the leak control extensions 2800. In some embodiments, the blood provides a force on the leak control extensions 2800 that causes the leak control extensions 2800 to pivot about the cap 114 or flex relative to the cap such that the blood moves around the leak control extensions 2800 in the direction X.

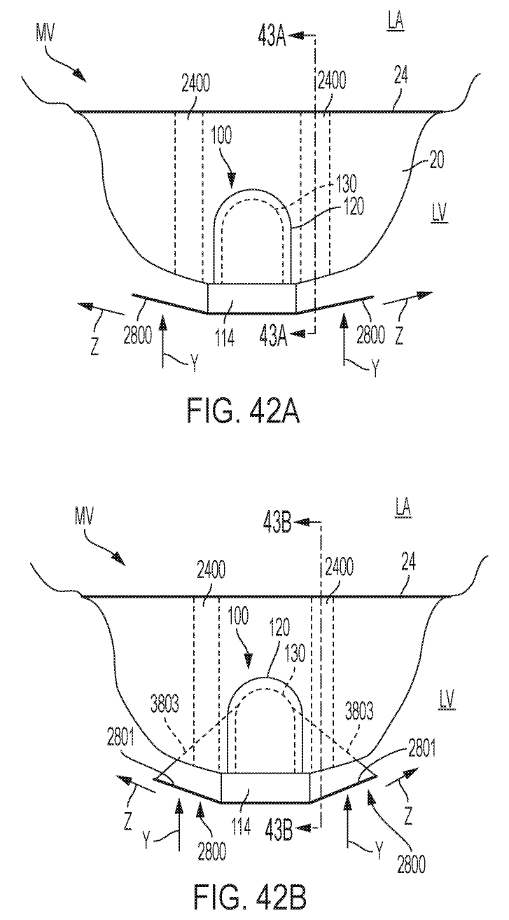

Referring to FIGS. 42A-45A, during the systolic phase, the leaflets 20, 22 of the mitral valve MV coapt to prevent blood from regurgitating into the left atrium LA as blood is pushed from the left ventricle and into the pulmonary artery PA, and the device 100 is connected to the leaflets 20, 22 to help facilitate the coapting or coaptation of the leaflets. In certain situations, however, the leaflets 20, 22 may not completely coapt around the device 100, which may cause one or more openings or jets 2400 to form between the device 100 and one or more of the leaflets 20, 22. The openings 2400 may form at the location where both leaflets 20, 22 and the device 100 meet. The openings 2400 can also form, however, at any point where the device 100 and a single leaflet 20, 22 meet. The leak control extensions 2800 can be positioned to prevent blood from regurgitating into the left atrium through the one or more openings 2400.

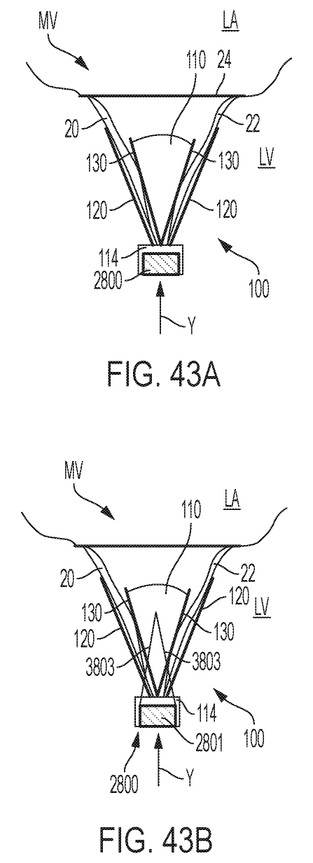

Referring to FIGS. 42A and 43A, contraction of the left ventricle LV pushes blood toward the mitral valve MV in the direction Y and engages the deflecting paddles 2801 of the leak control extensions 2800, which prevents blood from moving through the openings 2400 and into the left atrium LA. In some embodiments, the blood provides a force on the deflecting paddles 2801 that causes the deflecting paddles 2801 to pivot about the cap 114 or flex relative to the cap such that the blood moves around the leak control extensions 2800 in the direction Z.

Referring to FIGS. 46A and 47A, the device 100 shown in FIGS. 38A-45A can be placed within the mitral valve MV in a location near the annulus 24. Similar to the device 100 being located in a more central location within the mitral valve MV (as shown in FIGS. 38A-45A), openings 2400 may form where both leaflets 20, 22 and the device 100 meet. As shown in the illustrated embodiment, when the device 100 is placed near the annulus 24, the force on the mitral valve MV by the device 100 may cause the opening 2400 adjacent to the annulus 24 to have a deformed shape. In some embodiments, the leak control extensions 2800 are flexible such that forces applied to the leak control extensions cause the leak control extensions to compress. For example, as shown in FIGS. 46A and 47A, the leak control extension 2800 adjacent to the annulus 24 is pressed against the annulus 24 (or the side wall of the left ventricle LV), which causes the leak control extension 2800 to compress into a deformed shape. This compression of the leak control extensions 2800 allows the leak control extensions to cover the deformed opening 2400 that is adjacent to the annulus 24. The flexible leak control extensions 2800 are advantageous because they allow the device 100 to be positioned within the mitral valve MV at various positions. In addition, the flexible leak control extensions 2800 are advantageous because they are capable of taking a form needed to block blood-flow through any deformed openings caused by the connection between the device 100 and the leaflets 20, 22.

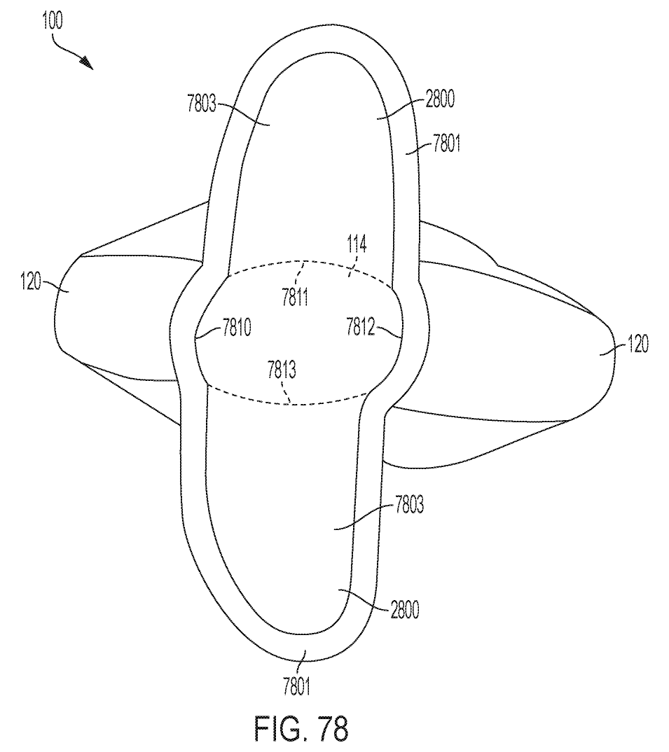

FIG. 78 illustrates a more specific example of the implantable prosthetic device 100 shown in FIGS. 38A-47A. The device 100 includes a pair of paddles 120, a pair of clasps (not shown), a coaption element (e.g., a spacer, etc.), a cap 114, and a pair of leak control extensions 2800. The leak control extensions 2800 have deflecting paddles 2801 that include a flexible frame 7801 and a barrier material 7803. The flexible frame 7801 can be made of, for example, a metal wire, such as a steel or nitinol wire, plastic, etc. The barrier material 7803 can take a wide variety of different forms. For example, the barrier material can be a cloth material, a biocompatible material, bovine or porcine heart tissue, a plastic membrane, etc. In the illustrated embodiment, the flexible frame 7801 of each leak control extension 2800 is attached to the front and rear edges 7810, 7812 of the cap 114, and the barrier material 7803 is optionally attached to the side edges 7811, 7813 of the cap 114. This connection between the leak control extensions 2800 and the cap 114 allows for the leak control extensions 2800 to pivot or flex about the side edges 7811, 7813 of the cap.

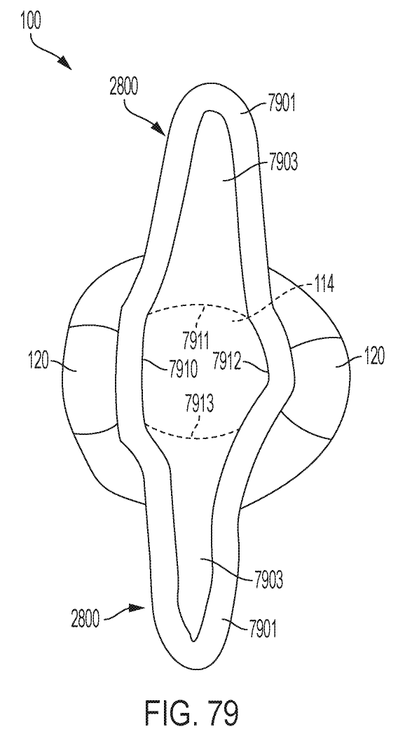

FIG. 79 illustrates a more specific example of the implantable prosthetic device 100 shown in FIGS. 38A-47A where the leak control extensions 2800 have a different shape than the FIG. 78 example. For example, the leak control extensions have a narrower shape. Though various example shapes are shown a variety of different shapes are possible, e.g., triangular, circular, rectangular, ovoid, oval, etc. In one example implementation, the shape of the leak control extensions 2800 can be modified by bending before the device 100 is implanted. The device 100 includes a pair of paddles 120, a pair of clasps (not shown), a coaption element (e.g., a spacer, etc.), a cap 114, and a pair of leak control extensions 2800. The leak control extensions 2800 have deflecting paddles 2801 that include a flexible frame 7901 and a barrier material 7903. The flexible frame 7901 can be made of, for example, a metal wire, such as a steel or nitinol wire, plastic, etc. In one example implementation, the flexible frame is made from a plastically deformable material to allow the shape of the flexible frame 7901 to be modified before implantation. The barrier material 7903 can take a wide variety of different forms. For example, the barrier material can be a cloth material, a biocompatible material, bovine or porcine heart tissue, a plastic membrane, etc. In the illustrated embodiment, the flexible frame 7901 of each leak control extension 2800 is attached to the front and rear edges 7910, 7912 of the cap 114, and the barrier material 7803 is optionally attached to the side edges 7911, 7913 of the cap 114. This connection between the leak control extensions 2800 and the cap 114 allows for the leak control extensions 2800 to pivot or flex about the side edges 7911, 7913 of the cap.

FIGS. 38B-47B show an example of an implantable prosthetic device 100 attached to leaflets 20, 22 of a native valve (illustrated, for example, as a mitral valve MV, but could be similarly used on another valve such as the tricuspid valve) in the fully closed position, where the device 100 includes one or more leak control extensions 2800. In the embodiment shown in FIGS. 38B-45B, the implantable prosthetic device 100 is attached to the native valve at a substantially central location within the annulus 24. It should be understood, however, that the implantable prosthetic device 100 can be attached to the leaflets 20, 22 at any location within the native valve (e.g., the location shown in FIGS. 46B-47B).

In some implementations, the device 100 has a pair of paddles 120, a pair of clasps 130, a coaption element 110 (e.g., a spacer etc.), a cap 114, and at least one leak control extension 2800. However, the implantable prosthetic device 100 can take any suitable form, such as, for example, any formed described in the present application in combination with at least one leak control extension 2800.

In some implementations, the leak control extension(s) 2800 are configured to prevent or inhibit blood from regurgitating from the ventricle and into the atrium during the systolic phase. That is, the leak control extension(s) 2800 extend from the device (e.g., from the end of the cap 114 of the device 100) such that the leak control extensions are positioned to prevent blood from moving through any jets or openings between the leaflets 20, 22 of the native valve during the systolic phase. In the illustrated embodiment, the leak control extensions 2800 include deflecting paddles 2801 that are positioned in the ventricle when the device 100 is attached to the leaflets 20, 22, where the deflecting paddles 2801 are attached to the spacer 110 by one or more arms 3803.

In some implementations, the arms 3803 secure the deflecting paddles 2801 to the spacer 110 to prevent the deflecting paddles 2801 from moving to a position that does not allow the deflecting paddles 2801 to prevent regurgitation of blood during the systolic phase. The arms 3803 can be made of, for example, a cloth material, a suture, a wire, any combination thereof, or any other suitable material or component that is capable of securing the deflecting paddles to the spacer 110. While the illustrated embodiment shows the arms 3803 connecting the deflecting paddles 2801 to the spacer 110, it should be understood that the arms 3803 can connect the deflecting paddles 2801 to any other portion of the device 100 that prevents unwanted movement of the deflecting paddles.

In the illustrated example, the device 100 has two leak control extensions 2800, where each leak control extension 2800 includes a deflecting paddle 2801 that is attached to a bottom surface of the cap 14. The deflecting paddles 2801 can be connected to each other (as shown in the illustrated embodiment), or the deflecting paddles 2801 can be separate extensions. While the illustrated embodiment is shown as having two leak control extensions 2800, it should be understood that the device 100 can have any suitable number of leak control extensions.

Referring to the example shown in FIGS. 40B and 41B, during the diastolic phase, the leaflets 20, 22 of the mitral valve MV open and blood moves from the left atrium LA to the left ventricle LV. As the device 100 is connected to the leaflets 20, 22, a portion 3003 of the mitral valve MV is substantially blocked by the device 100 when the leaflets 20, 22 are in the open position, while other portion(s) 3005 of the mitral valve are open such that blood can move into the left ventricle LV. The blood moves through the open portions 3005 in the direction D and engages the leak control extensions 2800. In some implementations, the blood provides a force on the leak control extensions 2800 that causes the deflecting paddles 2801 to pivot about the cap 114 or flex relative to the cap such that the blood moves around the leak control extensions 2800 in the direction X.

Referring to FIGS. 42B-45B, during the systolic phase, the leaflets 20, 22 of the mitral valve MV coapt to prevent blood from regurgitating into the left atrium LA as blood is pushed from the left ventricle and into the pulmonary artery PA, and the device 100 is connected to the leaflets 20, 22 to help facilitate the coapting or coaptation of the leaflets. In certain situations, however, the leaflets 20, 22 may not completely coapt around the device 100, which may cause one or more openings or jets 2400 to form between the device 100 and one or more of the leaflets 20, 22. The openings 2400 may form at the location where both leaflets 20, 22 and the device 100 meet. The openings 2400 can also form, however, at any point where the device 100 and a single leaflet 20, 22 meet. The leak control extensions 2800 can be positioned to prevent blood from regurgitating into the left atrium through the one or more openings 2400.

Referring to FIGS. 42B and 43B, contraction of the left ventricle LV pushes blood toward the mitral valve MV in the direction Y and engages the deflecting paddles 2801 of the leak control extensions 2800, which prevents blood from moving through the openings 2400 and into the left atrium LA. In some implementations, the blood provides a force on the deflecting paddles 2801 that causes the deflecting paddles 2801 to pivot about the cap 114 or flex relative to the cap such that the blood moves around the leak control extensions 2800 in the direction Z.

Referring to FIGS. 46B and 47B, the device 100 shown in FIGS. 38B-45B can be placed within the native valve in a location near the annulus 24. Similar to the device 100 being located in a more central location within the native valve (as shown in FIGS. 38B-45B), openings 2400 may form where both leaflets 20, 22 and the device 100 meet. As shown in the illustrated embodiment, when the device 100 is placed near the annulus 24, the force on the native valve by the device 100 may cause the opening 2400 adjacent to the annulus 24 to have a deformed shape. In some implementations, the leak control extensions 2800 are flexible such that forces applied to the leak control extensions cause the leak control extensions to compress. For example, as shown in FIGS. 46B and 47B, the leak control extension 2800 adjacent to the annulus 24 is pressed against the annulus 24 (or the side wall of the ventricle), which causes the leak control extension 2800 to compress into a deformed shape. This compression of the leak control extensions 2800 allows the leak control extensions to cover the deformed opening 2400 that is adjacent to the annulus 24. The flexible leak control extensions 2800 are advantageous because they allow the device 100 to be positioned within the native valve at various positions. In addition, the flexible leak control extensions 2800 are advantageous because they are capable of taking a form needed to block blood-flow through any deformed openings caused by the connection between the device 100 and the leaflets 20, 22.

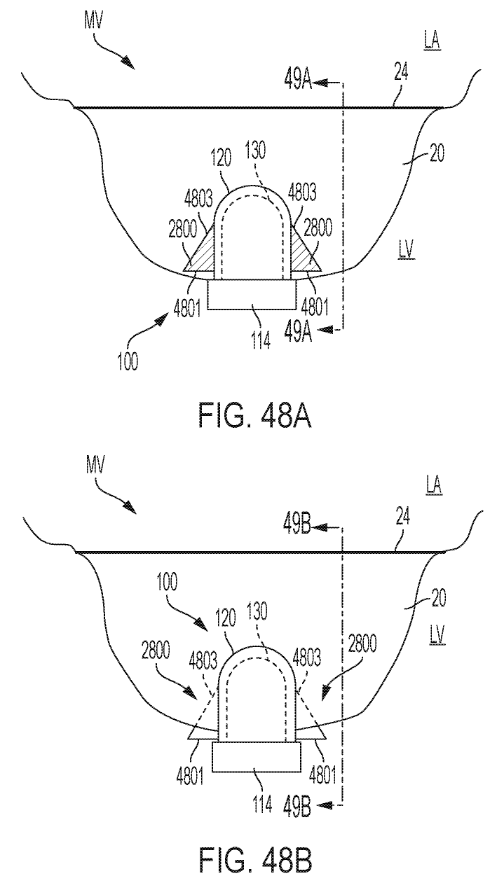

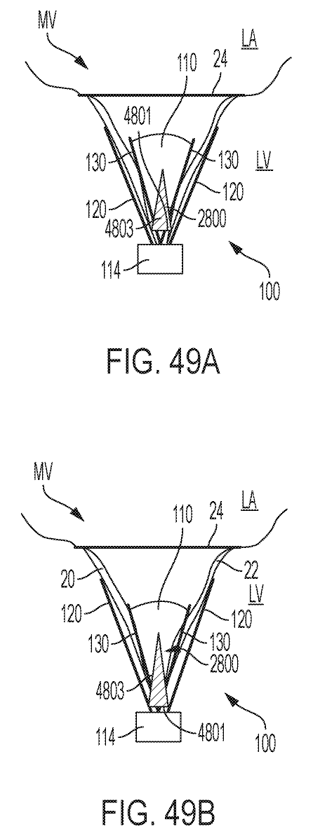

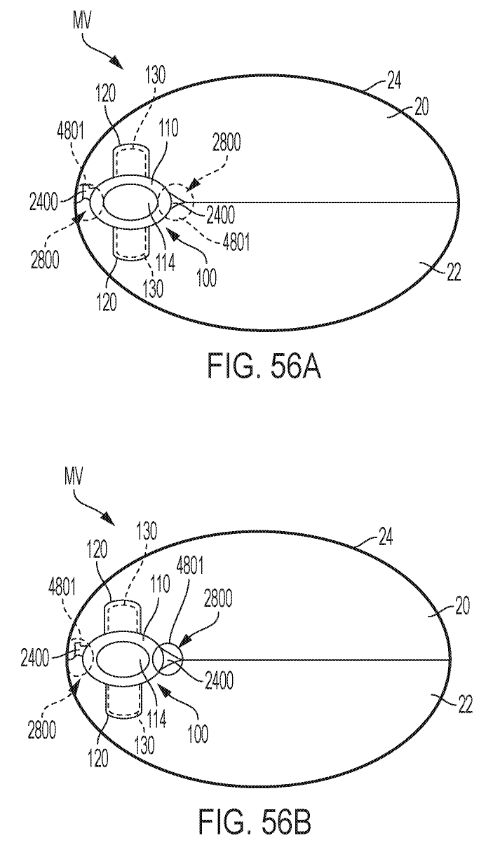

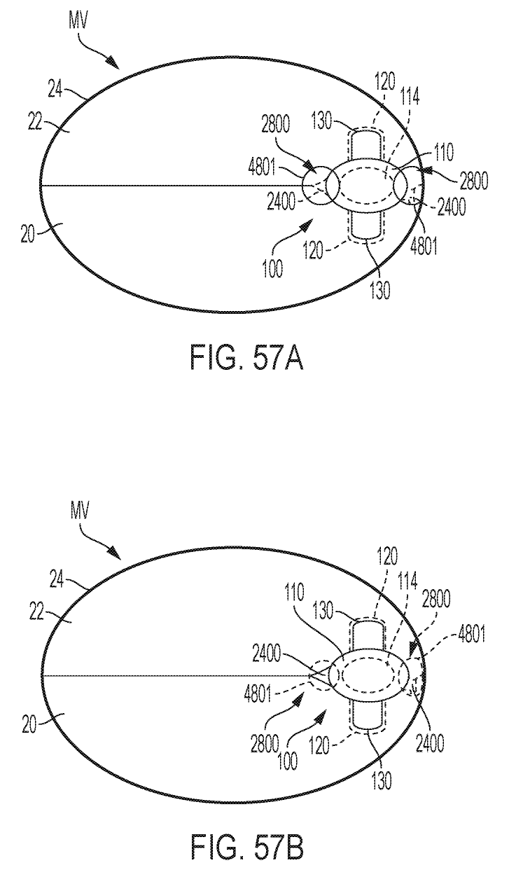

FIGS. 48A-57A show an example of an implantable prosthetic device 100 attached to the leaflets 20, 22 of a native valve (illustrated, for example, as mitral valve MV, but could be similarly used on another valve such as the tricuspid valve) in the fully closed position, where the device 100 includes one or more leak control extensions 2800. In the embodiment shown in FIGS. 48A-55A, the implantable prosthetic device 100 is attached to the native valve at a substantially central location, with the leak control extensions 2800 between the bottom of the native valve leaflets and the native valve annulus. It should be understood, however, that the implantable prosthetic device 100 can be attached to the leaflets 20, 22 at any location (e.g., the location shown in FIGS. 56A-57A). The device 100 has a pair of paddles 120, a pair of clasps 130, a coaption element 110 (e.g., a spacer, etc.), a cap 114, and at least one leak control extension 2800. However, the implantable prosthetic device 100 can take any suitable form, such as, for example, any formed described in the present application in combination with at least one leak control extension 2800.

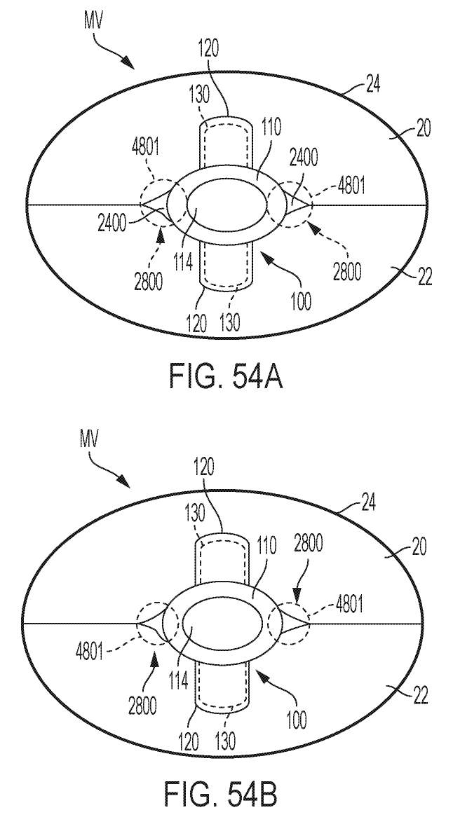

The leak control extension(s) 2800 are configured to prevent blood from regurgitating from the ventricle and into the atrium during the systolic phase. That is, the leak control extension(s) 2800 extend from the device such that the leak control extensions are positioned to block blood moving through openings between the leaflets 20, 22 of the native valve during the systolic phase. In the illustrated embodiment, the leak control extensions 2800 include pockets that are configured for receiving blood to prevent the blood from moving into the atrium. In some embodiments, the leak control extensions 2800 can have a flexible frame or loop 4801 that defines an opening of the pocket and a barrier material 4803 that defines the interior of the pocket. The flexible frame 4801 can have any suitable shape for defining the opening of the pocket, such as, for example, a circular shape (as shown in FIGS. 54A-57A), an oval shape, a triangular shape, a polygonal shape, etc. The flexible frame 4801 can be made of, for example, a metal wire, such as a steel or nitinol wire, plastic, etc. The barrier material 4803 is configured to capture the blood such that the blood does not move through the leak control extension 2800. The barrier material 4803 can be made of, for example, a cloth material, a biocompatible material, bovine or porcine heart tissue, a plastic membrane, etc.

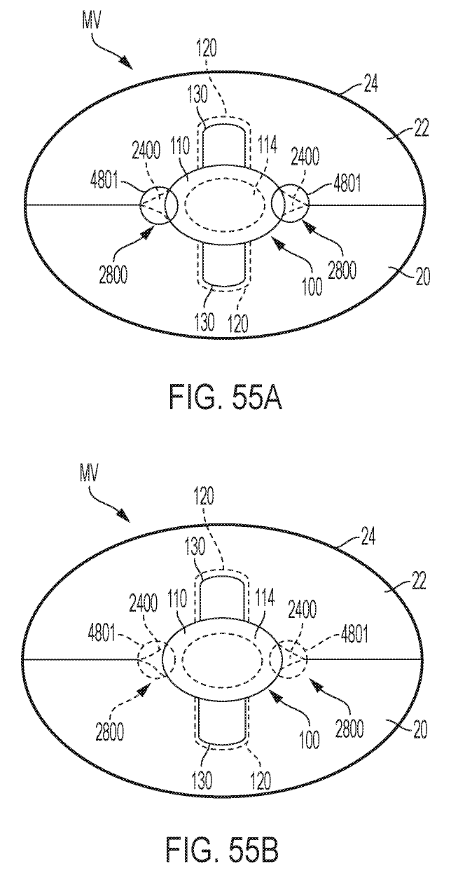

In the illustrated embodiment, the device 100 has two leak control extensions 2800 that are attached to the coaption element or spacer 110 (See FIG. 55A). In some embodiments, the opening of the leak control extensions 2800 are positioned in the atrium when the device 100 is attached to the leaflets 20, 22. The leak control extensions 2800 can, however, be attached to any other portion of the device 100 that allows the leak control extensions 2800 to be positioned to prevent blood from regurgitating into the atrium, and the opening of the leak control extensions 2800 can be in either the atrium or the ventricle. For example, the leak control extensions 2800 can be attached to the pair of paddles 120, the pair of clasps 130, the coaption element 110, the cap 114, or any combination thereof. While the illustrated embodiment is shown as having two leak control extensions 2800, it should be understood that the device 100 can have any suitable number of leak control extensions.

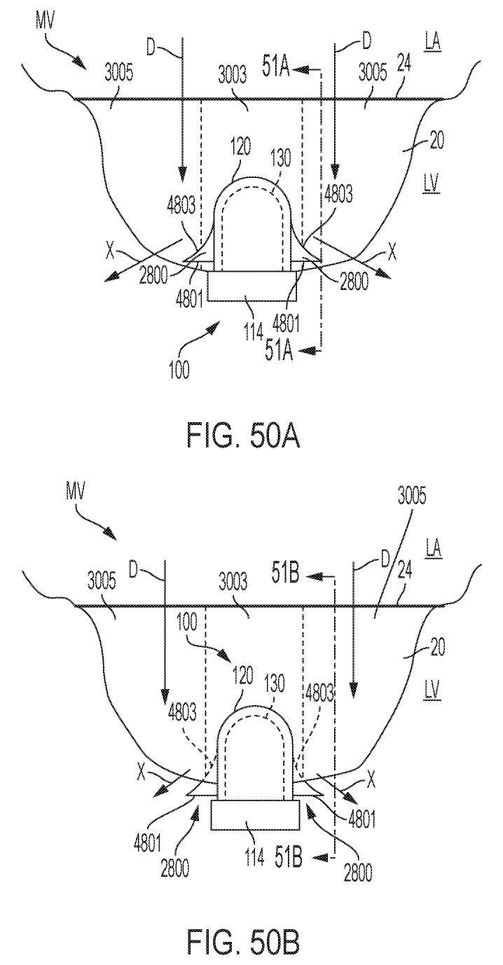

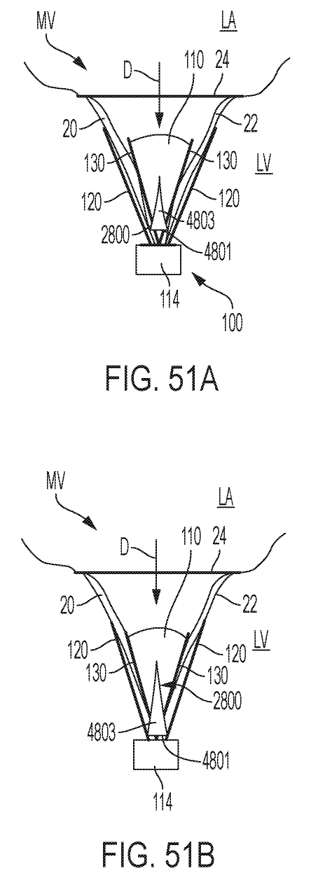

Referring to FIGS. 50A and 51A, during the diastolic phase, the leaflets 20, 22 of the mitral valve MV open and blood moves from the left atrium LA to the left ventricle LV. As the device 100 is connected to the leaflets 20, 22, a portion 3003 of the mitral valve MV is substantially blocked by the device 100 when the leaflets 20, 22 are in the open position, while other portion(s) 3005 of the mitral valve are open such that blood can move into the left ventricle LV. The blood moves through the open portions 3005 in the direction D and engages the leak control extensions 2800. In some embodiments, the blood provides a force on the barrier material 4803 of the leak control extensions 2800 that causes the barrier material 4803 to be compressed, and causes the blood to move around the leak control extensions 2800 in the direction X.

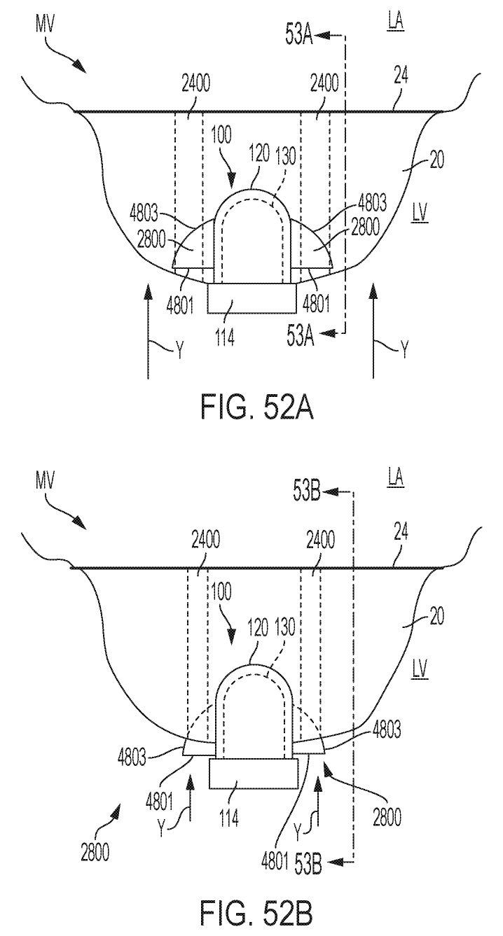

Referring to FIGS. 52A-55A, during the systolic phase, the leaflets 20, 22 of the mitral valve MV coapt to prevent blood from regurgitating into the left atrium LA as blood is pushed from the left ventricle and into the aorta, and the device 100 is connected to the leaflets 20, 22 to help facilitate the coapting or coaptation of the leaflets. In certain situations, however, the leaflets 20, 22 may not completely coapt around the device 100, which may cause one or more openings or jets 2400 to form between the device 100 and one or more of the leaflets 20, 22. The openings 2400 may form at the location where both leaflets 20, 22 and the device 100 meet. The openings 2400 can also form, however, at any point where the device 100 and a single leaflet 20, 22 meet. The leak control extensions 2800 are positioned to prevent blood from regurgitating into the left atrium through the one or more openings 2400.

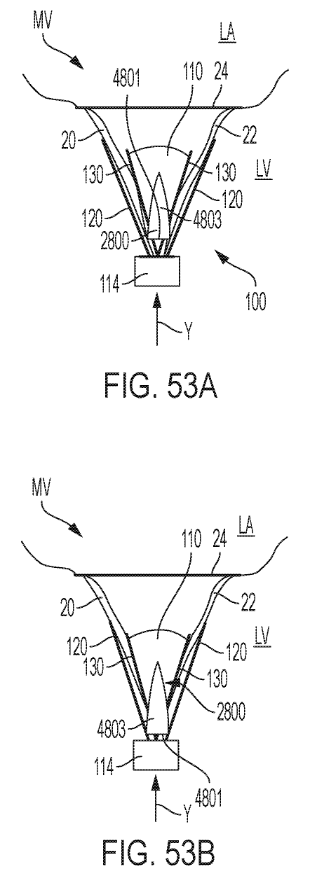

Referring to FIGS. 52A and 53A, contraction of the left ventricle LV pushes blood toward the mitral valve MV in the direction Y and engages the leak control extensions 2800 in the mitral valve, which prevents additional blood from moving through the openings 2400 and into the left atrium LA. In some embodiments, such as the illustrated embodiment, the blood enters the leak control extensions 2800 and provides a force on the barrier material 4803 that causes the barrier material 4803 to expand.

Referring to FIGS. 56A and 57A, the device 100 shown in FIGS. 48A-55A can be placed within the mitral valve MV in a location near the annulus 24. Similar to the device 100 being located in a more central location within the mitral valve MV (as shown in FIGS. 48A-55A), openings 2400 may form where both leaflets 20, 22 and the device 100 meet. As shown in the illustrated embodiment, when the device 100 is placed near the annulus 24, the force on the mitral valve MV by the device 100 may cause the opening 2400 adjacent to the annulus 24 to have a deformed shape. In some embodiments, the flexible frame 4801 of the leak control extensions 2800 are flexible such that forces applied to the leak control extensions 2800 cause the flexible frame 4801 to compress. For example, as shown in FIGS. 56A and 57A, the leak control extension 2800 adjacent to the annulus 24 is pressed against the annulus 24 (or the side wall of the left ventricle LV), which causes the flexible frame 4801 to compress into a deformed shape. This compression of the flexible frame 4801 allows the leak control extensions to cover the deformed opening 2400 that is adjacent to the annulus 24. The flexible leak control extensions 2800 are advantageous because it allows the device 100 to be positioned within the mitral valve MV at various locations. In addition, the flexible leak control extensions 2800 are advantageous because they are capable of taking the form of any deformed openings caused by the connection between the device 100 and the leaflets 20, 22.

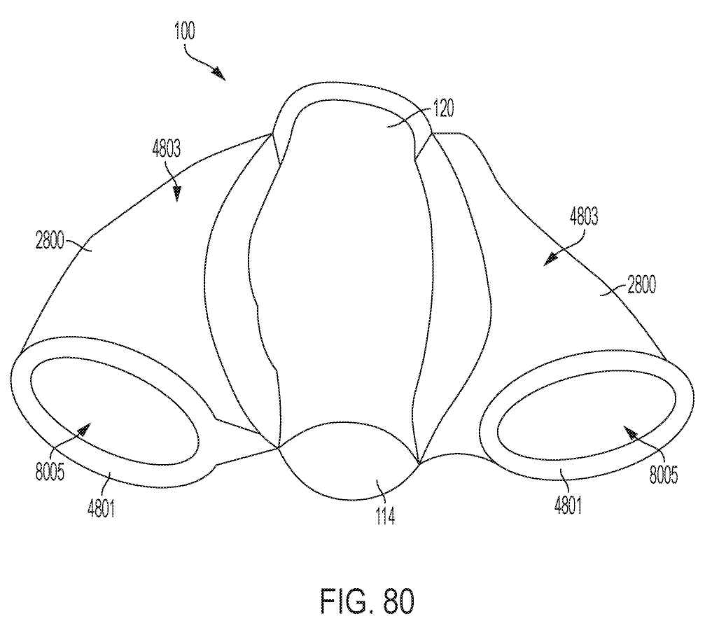

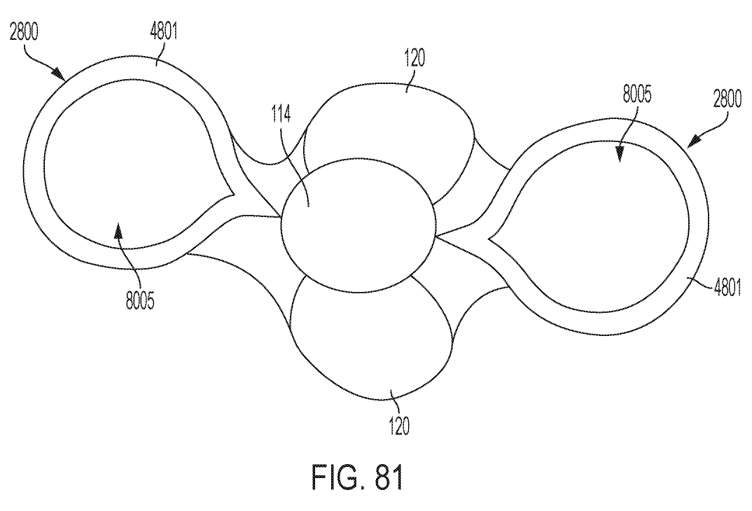

FIGS. 80 and 81 illustrate a more specific example of the implantable prosthetic device 100 shown in FIGS. 48A-57A. The device 100 includes a pair of paddles 120, a pair of clasps (not shown), a coaption element (e.g., a spacer, etc.), a cap 114, and a pair of leak control extensions 2800. The leak control extensions 2800 have a flexible frame 4801 that defines an opening 8005 of the pocket and a barrier material 4803 that defines the interior of the pocket. In the illustrated embodiment, the flexible frame 4801 is formed to give the opening 8005 a circular shape. The barrier material 4803 is configured to capture the blood such that the blood does not move through the leak control extension 2800.

FIGS. 48B-57B show an example implantable prosthetic device 100 attached to the leaflets 20, 22 of a native valve (illustrated, for example, as mitral valve MV, but could be similarly used on another valve such as the tricuspid valve) in the fully closed position, where the device 100 includes one or more leak control extensions 2800. In the embodiment shown in FIGS. 48B-55B, the implantable prosthetic device 100 is attached to the native valve at a substantially central location, with the leak control extensions 2800 between the bottom of the native valve leaflets and the native valve annulus. It should be understood, however, that the implantable prosthetic device 100 can be attached to the leaflets 20, 22 at any location (e.g., the location shown in FIGS. 56B-57B). The device 100 has a pair of paddles 120, a pair of clasps 130, a coaption element 110 (e.g., a spacer, etc.), a cap 114, and at least one leak control extension 2800. However, the implantable prosthetic device 100 can take any suitable form, such as, for example, any formed described in the present application in combination with at least one leak control extension 2800.

The leak control extension(s) 2800 are configured to prevent blood from regurgitating from the ventricle and into the atrium during the systolic phase. That is, the leak control extension(s) 2800 extend from the device such that the leak control extensions are positioned to block blood moving through openings between the leaflets 20, 22 of the native valve during the systolic phase. In the illustrated embodiment, the leak control extensions 2800 are pockets that are configured for receiving blood to prevent the blood from moving into the atrium. In some embodiments, the leak control extensions 2800 can have a flexible frame or loop 4801 that defines an opening of the pocket and a barrier material 4803 that defines the interior of the pocket. The flexible frame 4801 can have any suitable shape for defining the opening of the pocket, such as, for example, a circular shape (as shown in FIGS. 54B-57B), an oval shape, a triangular shape, a polygonal shape, etc. The flexible frame 4801 can be made of, for example, a metal wire, such as a steel or nitinol wire, plastic, etc. The barrier material 4803 is configured to capture the blood such that the blood does not move through the leak control extension 2800. The barrier material 4803 can be made of, for example, a cloth material, a biocompatible material, bovine or porcine heart tissue, a plastic membrane, etc.

In the illustrated embodiment, the device 100 has two leak control mechanisms or leak control extensions 2800 that are attached to the coaption element or spacer 110 (See FIG. 55B). In the illustrated embodiment, when the device 100 is attached to the leaflets 20, 22, a portion of the leak control extensions 2800 are in the atrium and another portion of the leak control extensions 2800 are in the ventricle such that the opening of the leak control extensions 2800 are positioned in the ventricle. The leak control extensions 2800 can, however, be attached to any other portion of the device 100 that allows the leak control extensions 2800 to be positioned to prevent blood from regurgitating into the atrium, and the opening of the leak control extensions 2800 can be in either the atrium or the ventricle. For example, the leak control extensions 2800 can be attached to the pair of paddles 120, the pair of clasps 130, the coaption element 110, the cap 114, or any combination thereof. While the illustrated embodiment is shown as having two leak control extensions 2800, it should be understood that the device 100 can have any suitable number of leak control extensions.

Referring to FIGS. 50B and 51B, during the diastolic phase, the leaflets 20, 22 of the mitral valve MV open and blood moves from the left atrium LA to the left ventricle LV. As the device 100 is connected to the leaflets 20, 22, a portion 3003 of the mitral valve MV is substantially blocked by the device 100 when the leaflets 20, 22 are in the open position, while other portion(s) 3005 of the mitral valve are open such that blood can move into the left ventricle LV. The blood moves through the open portions 3005 in the direction D and engages the leak control extensions 2800. In some embodiments, the blood provides a force on the barrier material 4803 of the leak control extensions 2800 that causes the barrier material 4803 to be compressed, and causes the blood to move around the leak control extensions 2800 in the direction X.

Referring to FIGS. 52B-55B, during the systolic phase, the leaflets 20, 22 of the mitral valve MV coapt to prevent blood from regurgitating into the left atrium LA as blood is pushed from the left ventricle LV and into the aorta, and the device 100 is connected to the leaflets 20, 22 to help facilitate the coapting or coaptation of the leaflets. In certain situations, however, the leaflets 20, 22 may not completely coapt around the device 100, which may cause one or more openings or jets 2400 to form between the device 100 and one or more of the leaflets 20, 22. The openings 2400 may form at the location where both leaflets 20, 22 and the device 100 meet. The openings 2400 can also form, however, at any point where the device 100 and a single leaflet 20, 22 meet. The leak control extensions 2800 are positioned to prevent blood from regurgitating into the left atrium through the one or more openings 2400.

Referring to FIGS. 52B and 53B, contraction of the left ventricle LV pushes blood toward the mitral valve MV in the direction Y and engages the leak control extensions 2800 in the mitral valve, which prevents additional blood from moving through the openings 2400 and into the left atrium LA. In some embodiments, such as the illustrated embodiment, the blood enters the leak control extensions 2800 and provides a force on the barrier material 4803 that causes the barrier material 4803 to expand.

Referring to FIGS. 56B and 57B, the device 100 shown in FIGS. 48B-55B can be placed within the native valve in a location near the annulus 24. Similar to the device 100 being located in a more central location within the native valve (as shown in FIGS. 54B-55B), openings 2400 may form where both leaflets 20, 22 and the device 100 meet. As shown in the illustrated embodiment, when the device 100 is placed near the annulus 24, the force on the native valve by the device 100 may cause the opening 2400 adjacent to the annulus 24 to have a deformed shape. In some embodiments, the flexible frame 4801 of the leak control extensions 2800 are flexible such that forces applied to the leak control extensions 2800 cause the flexible frame 4801 to compress. For example, as shown in FIGS. 56B and 57B, the leak control extension 2800 adjacent to the annulus 24 is pressed against the annulus 24 (or the side wall of the ventricle), which causes the flexible frame 4801 to compress into a deformed shape. This compression of the flexible frame 4801 allows the leak control extensions to cover the deformed opening 2400 that is adjacent to the annulus 24. The flexible leak control extensions 2800 are advantageous because it allows the device 100 to be positioned within the native valve at various locations. In addition, the flexible leak control extensions 2800 are advantageous because they are capable of taking the form of any deformed openings caused by the connection between the device 100 and the native leaflets.

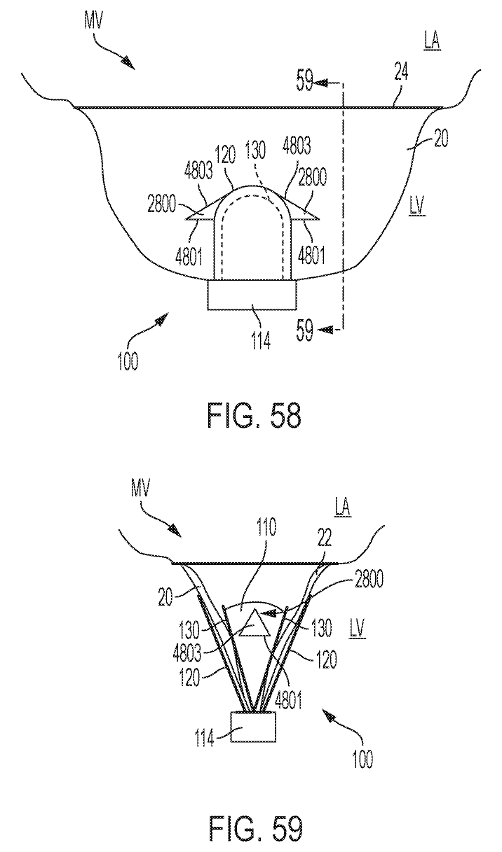

FIGS. 58-67 show an example of an implantable prosthetic device 100 attached to leaflets 20, 22 of a native valve (illustrated, for example, as mitral valve MV, but could be similarly used on another valve such as the tricuspid valve). In the embodiment shown in FIGS. 58-65, the implantable prosthetic device 100 is attached to the native valve at a substantially central location of the valve leaflets. It should be understood, however, that the implantable prosthetic device 100 can be attached to the leaflets 20, 22 at any location (e.g., the location shown in FIGS. 56A-57A). In the example illustrated by FIGS. 58-67, the leak control extensions 2800 are positioned between the ends of the native valve leaflets and the native valve annulus. The device 100 has a pair of paddles 120, a pair of clasps 130, a coaption element 110 (e.g., a spacer, etc.), a cap 114, and at least one leak control extension 2800. However, the implantable prosthetic device 100 can take any suitable form, such as, for example, any formed described in the present application in combination with at least one leak control extension 2800.

The leak control extension(s) 2800 are configured to prevent blood from regurgitating from the ventricle and into the atrium during the systolic phase. In the example illustrated by FIGS. 58-67, the leak control extension(s) 2800 extend from the device such that the leak control extensions are positioned to block blood that flows through the openings between the leaflets 20, 22 of the native valve during the systolic phase. In the illustrated embodiment, the leak control extensions 2800 are pockets that are configured for receiving blood to block blood in the native valve that is moving toward the atrium.

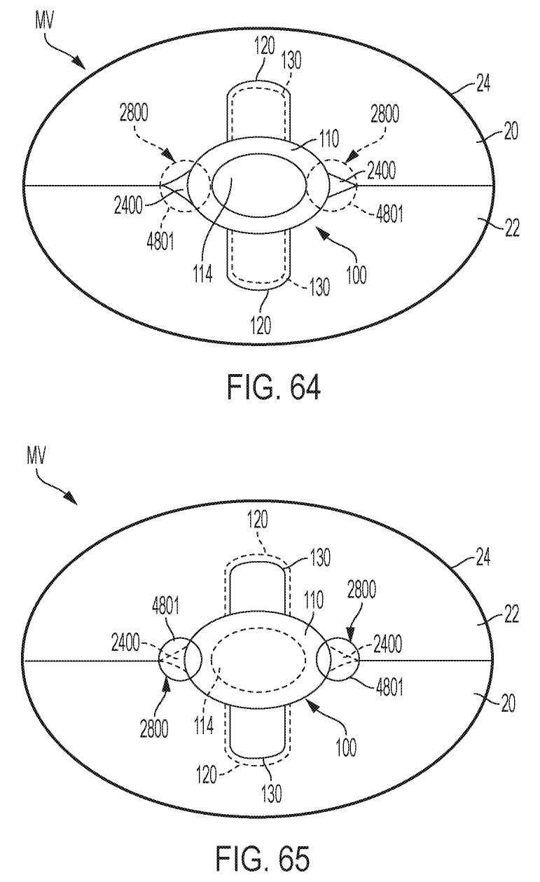

In some embodiments, the leak control extensions 2800 can have a flexible frame or loop 4801 that defines an opening of the pocket and a barrier material 4803 that defines the interior of the pocket. The flexible frame 4801 can have any suitable shape for defining the opening of the pocket, such as, for example, a circular shape (as shown in FIGS. 64-67), an oval shape, a triangular shape, a polygonal shape, etc. The flexible frame 4801 can be made of, for example, a metal wire, such as a steel or nitinol wire, plastic, etc. The barrier material 4803 is configured to capture the blood such that the blood does not move through the leak control extension 2800. The barrier material 4803 can be made of, for example, a cloth material, a biocompatible material, bovine or porcine heart tissue, a plastic membrane, etc.

In the illustrated embodiment, the device 100 has two leak control extensions 2800 that are attached to the coaption element or spacer 110 at a top portion of the device 100. In some embodiments, the opening of the leak control extensions 2800 are positioned about midway between the native valve annulus and the ends of the native valve leaflets when the device 100 is attached to the leaflets 20, 22. The leak control extensions 2800 can be attached to any other portion of the device 100 that allows the leak control extensions 2800 to be positioned to block all or a portion of blood from regurgitating into the atrium, and the opening of the leak control extensions 2800 can be in either the atrium or the ventricle. For example, the leak control extensions 2800 can be attached to the pair of paddles 120, the pair of clasps 130, the coaption element 110, the cap 114, or any combination thereof. In addition, the leak control extensions 2800 can be positioned at any position along the height of the device 100. While the illustrated embodiment is shown as having two leak control extensions 2800, it should be understood that the device 100 can have any suitable number of leak control extensions.

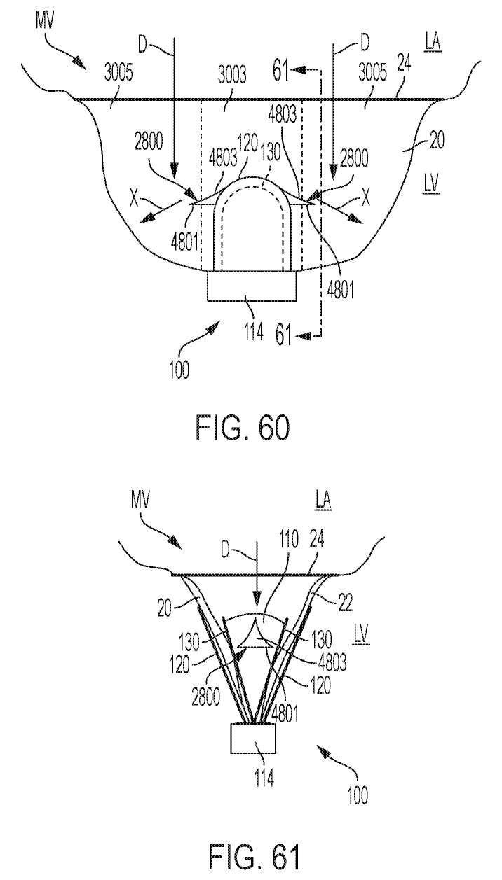

Referring to FIGS. 60 and 61, during the diastolic phase, the leaflets 20, 22 of the native valve (shown as mitral valve MV) open and blood moves from the left atrium LA to the left ventricle LV. As the device 100 is connected to the leaflets 20, 22, a portion 3003 of the mitral valve MV is substantially blocked by the device 100 when the leaflets 20, 22 are in the open position, while other portion(s) 3005 of the mitral valve are open such that blood can move into the left ventricle LV. The blood moves through the open portions 3005 in the direction D and engages the leak control extensions 2800. In some embodiments, the blood provides a force on the barrier material 4803 of the leak control extensions 2800 that causes the barrier material 4803 to be compressed, and causes the blood to move around the leak control extensions 2800 in the direction X.

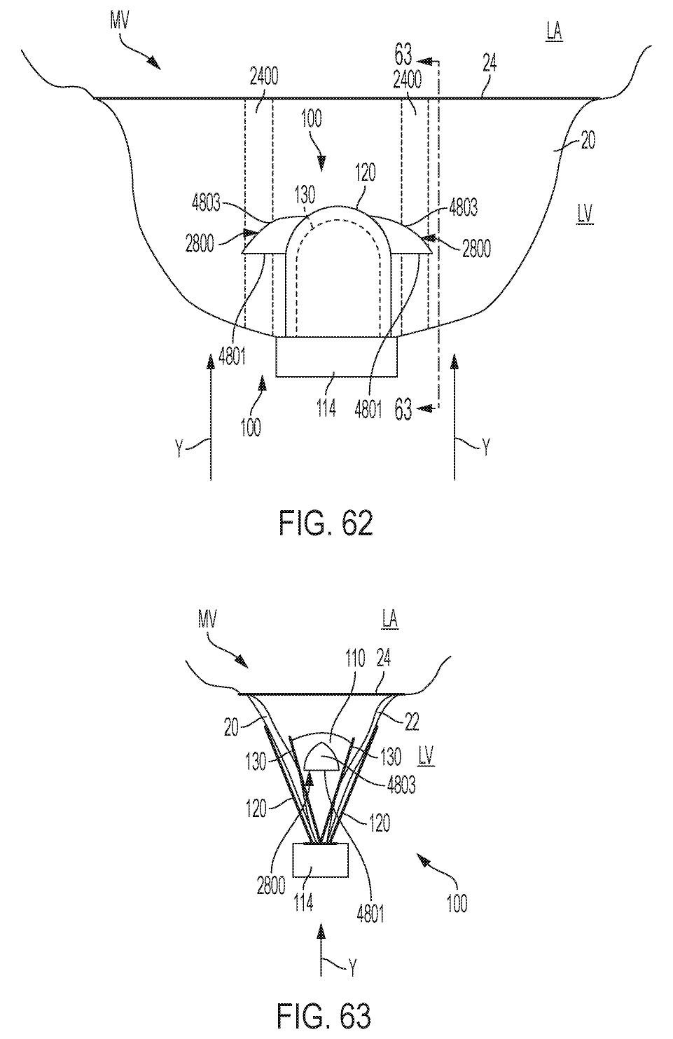

Referring to FIGS. 62-65, during the systolic phase, the leaflets 20, 22 of the mitral valve MV coapt to prevent blood from regurgitating into the left atrium LA as blood is pushed from the left ventricle and into the pulmonary artery PA, and the device 100 is connected to the leaflets 20, 22 to help facilitate the coapting or coaptation of the leaflets. In certain situations, however, the leaflets 20, 22 may not completely coapt around the device 100, which may cause one or more openings or jets 2400 to form between the device 100 and one or more of the leaflets 20, 22. The openings 2400 may form at the location where both leaflets 20, 22 and the device 100 meet. The openings 2400 can also form, however, at any point where the device 100 and a single leaflet 20, 22 meet. The leak control extensions 2800 are positioned to block blood that regurgitates through the one or more openings 2400 from flowing into the left atrium.

Referring to FIGS. 62 and 63, contraction of the left ventricle LV pushes blood toward the mitral valve MV in the direction Y, through the openings 2400 and into engagement with the leak control extensions 2800. This engagement with the leak control extensions 2800 prevents the blood that flows through the openings 2400 from flowing into the left atrium LA. In some embodiments, such as the illustrated embodiment, the blood enters the leak control extensions 2800 and provides a force on the barrier material 4803 that causes the barrier material 4803 to expand.

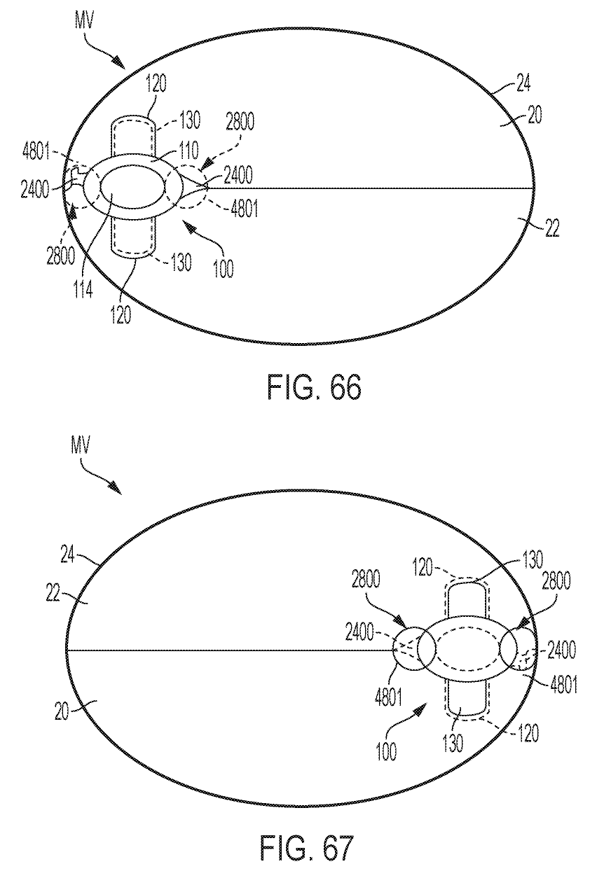

Referring to FIGS. 66 and 67, the device 100 shown in FIGS. 58-65 can be placed within the native valve in a location near the annulus 24. Similar to the device 100 being located in a more central location within the native valve (as shown in FIGS. 58-65), openings 2400 may form where both leaflets 20, 22 and the device 100 meet. As shown in the illustrated embodiment, when the device 100 is placed near the annulus 24, the force on the native valve by the device 100 may cause the opening 2400 adjacent to the annulus 24 to have a deformed shape. In some embodiments, the flexible frame 4801 of the leak control extensions 2800 are flexible such that forces applied to the leak control extensions 2800 cause the flexible frame 4801 to compress. For example, as shown in FIGS. 66 and 67, the leak control extension 2800 adjacent to the annulus 24 is pressed against the annulus 24 (or the side wall of the ventricle), which causes the flexible frame 4801 to compress into a deformed shape. This compression of the flexible frame 4801 allows the leak control extensions to cover the deformed opening 2400 that is adjacent to the annulus 24. The flexible leak control extensions 2800 are advantageous because they allow the device 100 to be positioned within the native valve at various locations without causing irritation to the annulus 24 or the side walls of the ventricle or the atrium. In addition, the flexible leak control extensions 2800 are advantageous because they are capable of taking a form that can block flow of any deformed openings caused by the connection between the device 100 and the leaflets 20, 22.

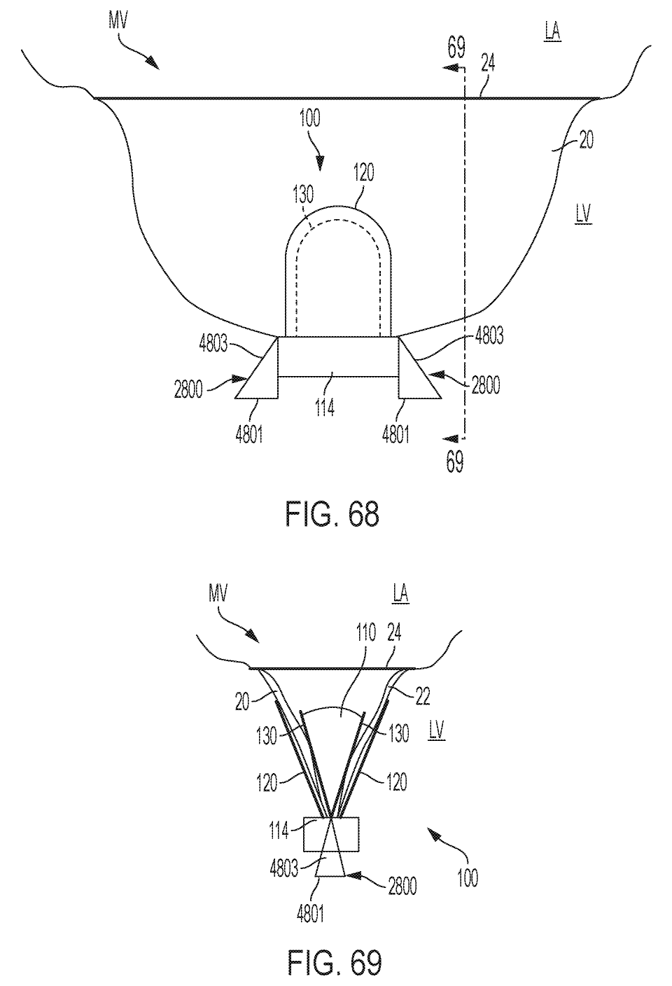

FIGS. 68-77 show an example of an implantable prosthetic device 100 attached to leaflets 20, 22 of a native valve (illustrated, for example, as mitral valve MV, but could be similarly used on another valve such as the tricuspid valve) in the fully closed position. In the embodiment shown in FIGS. 68-75, the implantable prosthetic device 100 is attached to the native valve at a substantially central location within the annulus 24. It should be understood, however, that the implantable prosthetic device 100 can be attached to the leaflets 20, 22 at any location within the native valve (e.g., the location shown in FIGS. 66-67). The device 100 has a pair of paddles 120, a pair of clasps 130, a coaption element 110 (e.g., spacer, etc.), a cap 114, and at least one leak control extension 2800. However, the implantable prosthetic device 100 can take any suitable form, such as, for example, any formed described in the present application in combination with at least one leak control extension 2800.

The leak control extension(s) 2800 are configured to prevent blood from regurgitating from the ventricle and into the atrium during the systolic phase. That is, the leak control extension(s) 2800 extend from the device such that the leak control extensions are positioned to prevent blood from moving through any jets or openings between the leaflets 20, 22 of the native valve during the systolic phase. In the illustrated embodiment, the leak control extensions 2800 are pockets that are configured for receiving blood to prevent the blood from moving into the atrium. In some embodiments, the leak control extensions 2800 have a flexible frame 4801 that defines an opening of the pocket and a barrier material 4803 that defines the interior of the pocket.

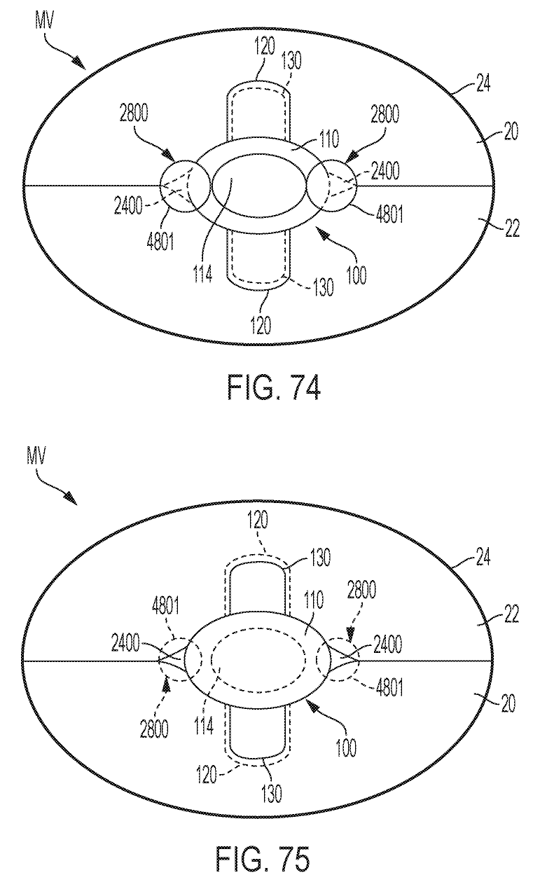

The flexible frame 4801 can have any suitable shape for defining the opening of the pocket, such as, for example, a circular shape (as shown in FIGS. 74-77), an oval shape, a triangular shape, a polygonal shape, etc. The flexible frame 4801 can be made of, for example, a metal wire, such as a steel or nitinol wire, plastic, etc. The barrier material 4803 is configured to capture the blood such that the blood does not move through the leak control extension 2800. The barrier material 4803 can be made of, for example, a cloth material, a biocompatible material, bovine or porcine heart tissue, a plastic membrane, etc. In the illustrated embodiment, the device 100 has two leak control extensions 2800 that are attached to the cap 114.

In some embodiments, the opening of the leak control extensions 2800 are positioned in the ventricle when the device 100 is attached to the leaflets 20, 22. The leak control extensions 2800 can be attached to any other portion of the device 100 that allows the leak control extensions 2800 to be positioned to prevent blood from regurgitating into the atrium, and the opening of the leak control extensions 2800 can be in either the atrium or the ventricle. For example, the leak control extensions 2800 can be attached to the pair of paddles 120, the pair of clasps 130, the coaption element 110, the cap 114, or any combination thereof. In addition, the leak control extensions 2800 can be positioned at any position along the height of the device 100. While the illustrated embodiment is shown as having two leak control extensions 2800, it should be understood that the device 100 can have any suitable number of leak control extensions.

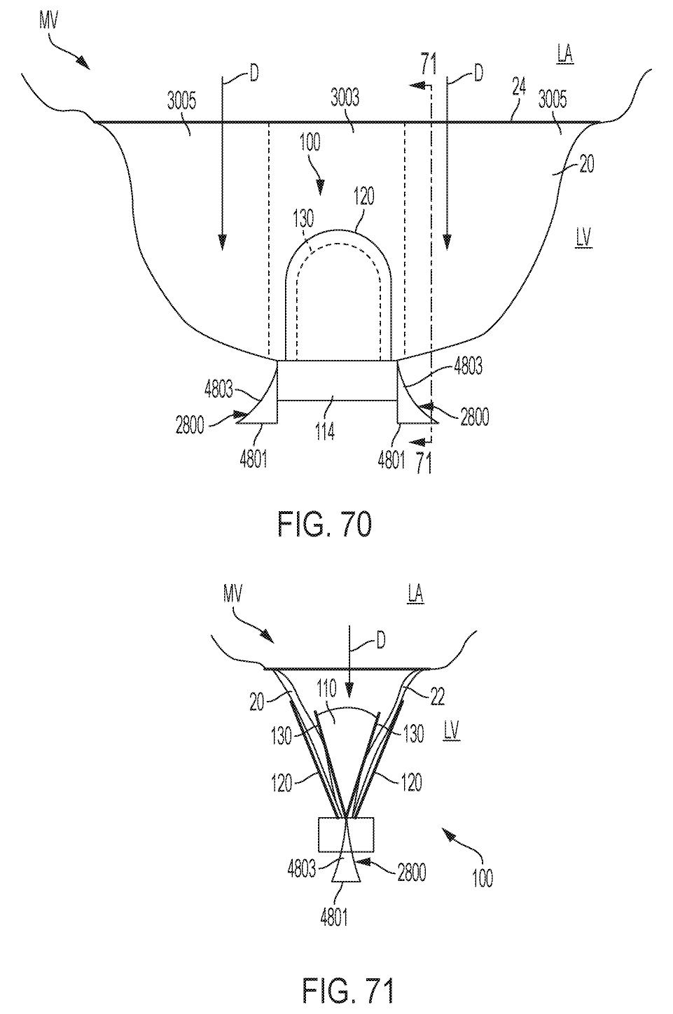

Referring to FIGS. 70 and 71, during the diastolic phase, the leaflets 20, 22 of the native valve (illustrated as mitral valve MV) open and blood moves from the left atrium LA to the left ventricle LV. As the device 100 is connected to the leaflets 20, 22, a portion 3003 of the mitral valve MV is substantially blocked by the device 100 when the leaflets 20, 22 are in the open position, while other portion(s) 3005 of the mitral valve are open such that blood can move into the left ventricle LV. The blood moves through the open portions 3005 in the direction D and engages the leak control extensions 2800. In some embodiments, the blood provides a force on the barrier material 4803 of the leak control extensions 2800 that causes the barrier material 4803 to be compressed, and causes the blood to move around the leak control extensions 2800 in the direction X.

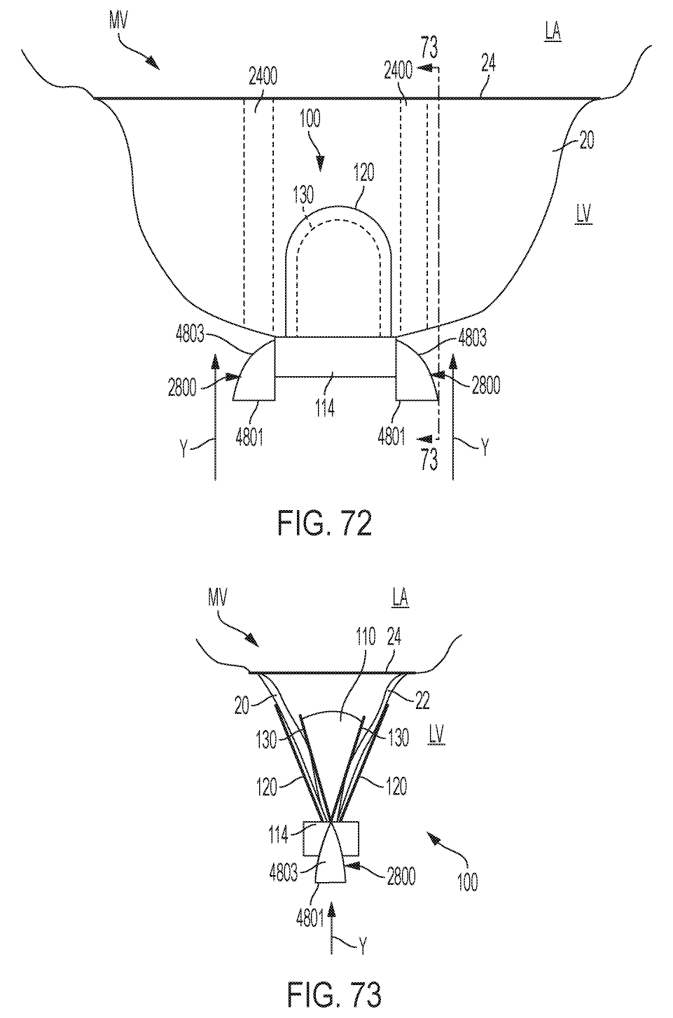

Referring to FIGS. 72-75, during the systolic phase, the leaflets 20, 22 of the mitral valve MV coapt to prevent blood from regurgitating into the left atrium LA as blood is pushed from the left ventricle and into the pulmonary artery PA, and the device 100 is connected to the leaflets 20, 22 to help facilitate the coapting or coaptation of the leaflets. In certain situations, however, the leaflets 20, 22 may not completely coapt around the device 100, which may cause one or more openings or jets 2400 to form between the device 100 and one or more of the leaflets 20, 22. The openings 2400 may form at the location where both leaflets 20, 22 and the device 100 meet. The openings 2400 can also form, however, at any point where the device 100 and a single leaflet 20, 22 meet. The leak control extensions 2800 are positioned to prevent blood from regurgitating into the left atrium through the one or more openings 2400.

Referring to FIGS. 72 and 73, contraction of the left ventricle LV pushes blood toward the mitral valve MV in the direction Y and engages the leak control extensions 2800, which prevents blood from moving through the openings 2400 and into the left atrium LA. In some embodiments, such as the illustrated embodiment, the blood enters the leak control extensions 2800 and provides a force on the barrier material 4803 that causes the barrier material 4803 to expand.

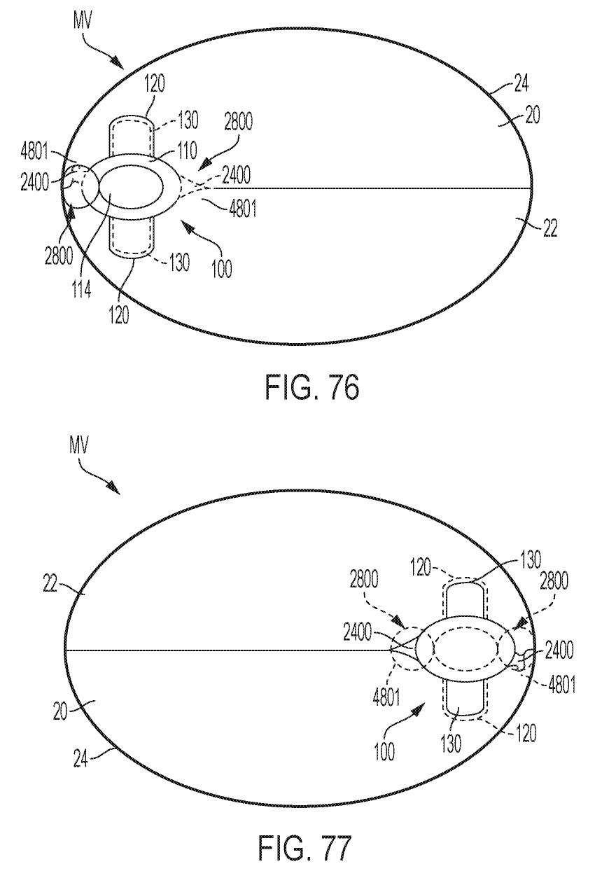

Referring to FIGS. 76 and 77, the device 100 shown in FIGS. 68-75 can be placed within the native valve in a location near the annulus 24. Similar to the device 100 being located in a more central location within the native valve (as shown in FIGS. 68-75), openings 2400 may form where both leaflets 20, 22 and the device 100 meet. As shown in the illustrated embodiment, when the device 100 is placed near the annulus 24, the force on the native valve by the device 100 may cause the opening 2400 adjacent to the annulus 24 to have a deformed shape.

In some embodiments, the flexible frame 4801 of the leak control extensions 2800 are flexible such that forces applied to the leak control extensions 2800 cause the flexible frame 4801 to compress or deform. For example, as shown in FIGS. 76 and 77, the leak control extension 2800 adjacent to the annulus 24 is pressed against the annulus 24 (or the side wall of the ventricle), which causes the flexible frame 4801 to compress into a deformed shape. This compression of the flexible frame 4801 allows the leak control extensions to cover the deformed opening 2400 that is adjacent to the annulus 24. The flexible leak control extensions 2800 are advantageous because it allows the device 100 to be positioned within the native valve at various locations without causing irritation to the annulus 24 or the side walls of the left ventricle or the atrium. In addition, the flexible leak control extensions 2800 are advantageous because they are capable of taking the form of any deformed openings caused by the connection between the device 100 and the leaflets 20, 22.

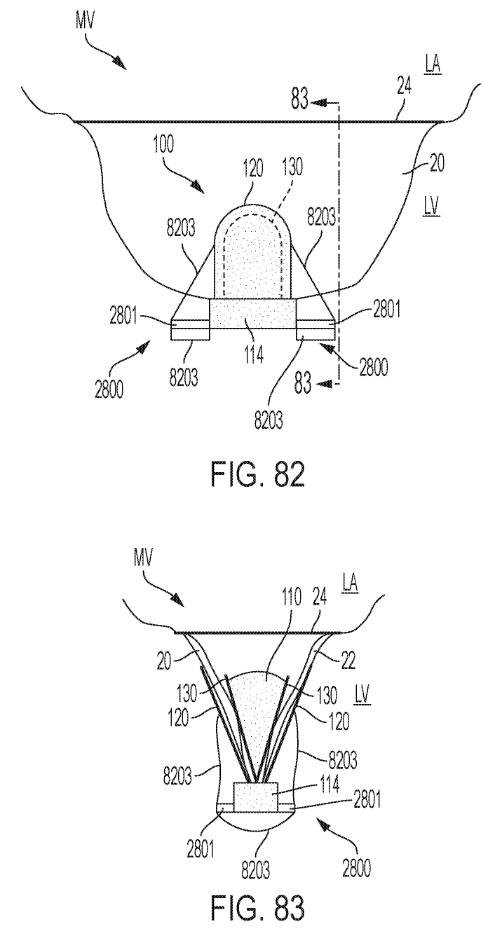

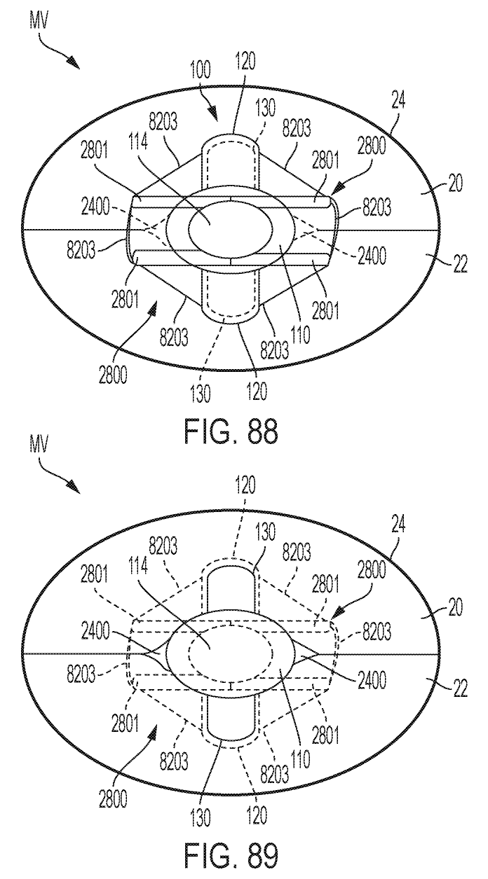

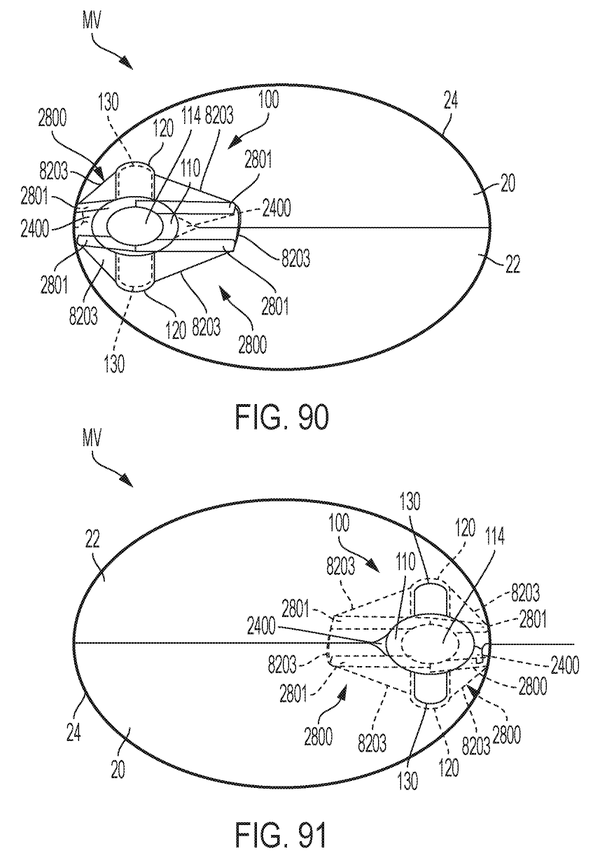

FIGS. 82-91 show an example of an implantable prosthetic device 100 attached to leaflets 20, 22 of a native valve (illustrated, for example, as mitral valve MV, but could be similarly used on another valve such as the tricuspid valve) in the fully closed position, where the device 100 includes one or more leak control extensions 2800. In the embodiment shown in FIGS. 82-89, the implantable prosthetic device 100 is attached to the native valve at a substantially central location within the annulus 24. It should be understood, however, that the implantable prosthetic device 100 can be attached to the leaflets 20, 22 at any location within the native valve (e.g., the location shown in FIGS. 90-91). The device 100 has a pair of paddles 120, a pair of clasps 130, a coaption element 110 (e.g., a spacer etc.), a cap 114, and at least one leak control extension 2800. However, the implantable prosthetic device 100 can take any suitable form, such as, for example, any formed described in the present application in combination with at least one leak control extension 2800.

The leak control extension(s) 2800 are configured to prevent blood from regurgitating from the ventricle and into the atrium during the systolic phase. That is, the leak control extension(s) 2800 extend from the device (e.g., from the end of the cap 114 of the device 100) such that the leak control extensions are positioned to prevent blood from moving through any jets or openings between the leaflets 20, 22 of the native valve during the systolic phase. Each of the leak control extensions 2800 can include one or more deflecting paddles 2801 (or other extension members) and a barrier element 8203. The one or more deflecting paddles 2801 can attach to the cap 114 of the device 100 (or any other portion of the device), and the barrier element 8203 can attach to the paddles 120 and the deflecting paddles 2801 to create a barrier that extends from a first paddle 120 of the device 100 to a second paddle of the device 100, where the barrier prevents regurgitation of blood during the systolic phase. In the illustrated embodiment, the leak control extensions 2800 each include two deflecting paddles 2801 that are attached to the cap 114 and a barrier element 8203 that connects to and extends from a first paddle 120, the two deflecting paddles 2801, and to the other paddle 120. The barrier element 8203 can include one or more pieces of material, such as, for example, one or more pieces of a cloth material, a biocompatible material, bovine or porcine heart tissue, a plastic membrane, etc. In the illustrated embodiment, the device 100 has two leak control extensions 2800, where each leak control extension 2800 includes one or more deflecting paddles 2801 and a barrier material 8203. While the illustrated embodiment is shown as having two leak control extensions 2800, it should be understood that the device 100 can have any suitable number of leak control extensions.

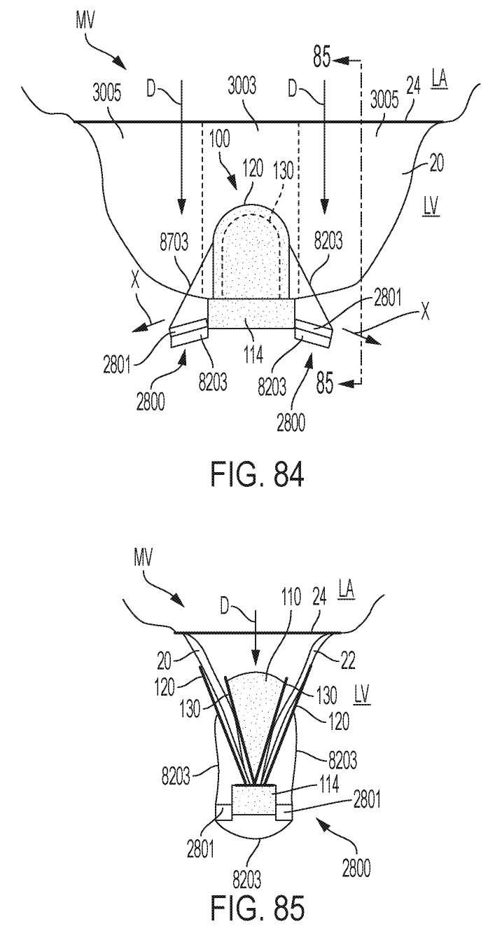

Referring to FIGS. 84 and 85, during the diastolic phase, the leaflets 20, 22 of the mitral valve MV open and blood moves from the left atrium LA to the left ventricle LV. As the device 100 is connected to the leaflets 20, 22, a portion 3003 of the mitral valve MV is substantially blocked by the device 100 when the leaflets 20, 22 are in the open position, while other portion(s) 3005 of the mitral valve are open such that blood can move into the left ventricle LV. The blood moves through the open portions 3005 in the direction D and engages the leak control extensions 2800. In some embodiments, the blood provides a force on the leak control extensions 2800 that causes the deflecting paddles 2801 to pivot about the cap 114 or flex relative to the cap such that the blood moves around the leak control extensions 2800 in the direction X.

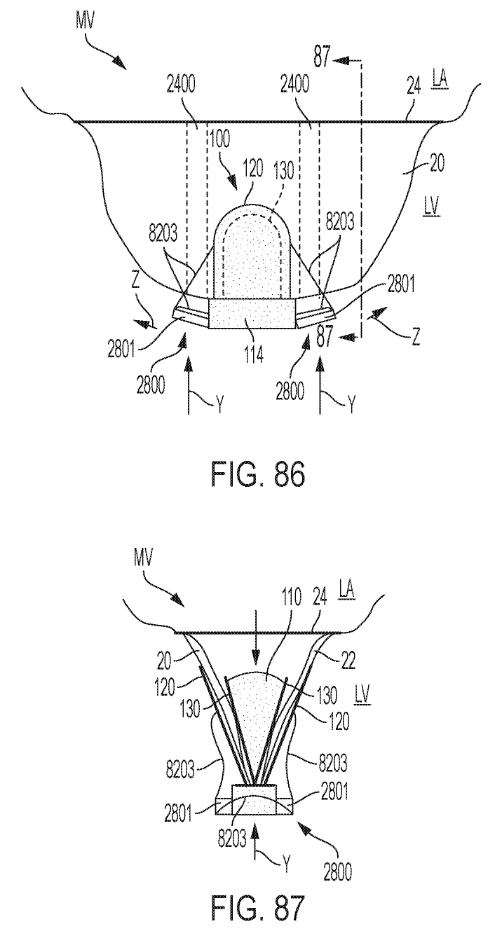

Referring to FIGS. 86-89, during the systolic phase, the leaflets 20, 22 of the mitral valve MV coapt to prevent blood from regurgitating into the left atrium LA as blood is pushed from the left ventricle and into the pulmonary artery PA, and the device 100 is connected to the leaflets 20, 22 to help facilitate the coapting or coaptation of the leaflets. In certain situations, however, the leaflets 20, 22 may not completely coapt around the device 100, which may cause one or more openings or jets 2400 to form between the device 100 and one or more of the leaflets 20, 22. The openings 2400 may form at the location where both leaflets 20, 22 and the device 100 meet. The openings 2400 can also form, however, at any point where the device 100 and a single leaflet 20, 22 meet. The leak control extensions 2800 can be positioned to prevent blood from regurgitating into the left atrium through the one or more openings 2400.

Referring to FIGS. 86 and 87, contraction of the left ventricle LV pushes blood toward the mitral valve MV in the direction Y and engages the deflecting paddles 2801 and barrier element 8203 of the leak control extensions 2800, which prevents blood from moving through the openings 2400 and into the left atrium LA. In some embodiments, the blood provides a force on the deflecting paddles 2801 that causes the deflecting paddles 2801 to pivot about the cap 114 or flex relative to the cap such that the blood moves around the leak control extensions 2800 in the direction Z. In some embodiments, the blood provides a force on the barrier element 8203 that causes a portion of the barrier element 8203 positioned between the two deflecting paddles 2801 to move in an upward direction.

Referring to FIGS. 90 and 91, the device 100 shown in FIGS. 82-89 can be placed within the native valve in a location near the annulus 24. Similar to the device 100 being located in a more central location within the native valve (as shown in FIGS. 82-89), openings 2400 may form where both leaflets 20, 22 and the device 100 meet. As shown in the illustrated embodiment, when the device 100 is placed near the annulus 24, the force on the native valve by the device 100 may cause the opening 2400 adjacent to the annulus 24 to have a deformed shape. In some embodiments, the leak control extensions 2800 are flexible such that forces applied to the leak control extensions cause the leak control extensions to compress. For example, as shown in FIGS. 90 and 91, the leak control extension 2800 adjacent to the annulus 24 is pressed against the annulus 24 (or the side wall of the ventricle), which causes the leak control extension 2800 to compress into a deformed shape. This compression of the leak control extensions 2800 allows the leak control extensions to cover the deformed opening 2400 that is adjacent to the annulus 24. The flexible leak control extensions 2800 are advantageous because they allow the device 100 to be positioned within the native valve at various positions. In addition, the flexible leak control extensions 2800 are advantageous because they are capable of taking a form needed to block blood-flow through any deformed openings caused by the connection between the device 100 and the leaflets 20, 22.

While various inventive aspects, concepts and features of the disclosures may be described and illustrated herein as embodied in combination in the example embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. Further, the techniques, methods, operations, steps, etc. described or suggested herein can be performed on a living animal or on a non-living simulation, such as on a cadaver, cadaver heart, simulator (e.g. with the body parts, tissue, etc. being simulated), etc. The words used in the claims have their full ordinary meanings and are not limited in any way by the description of the embodiments in the specification.

1. A valve repair device for repairing a native valve of a patient, the valve repair device comprising:
an anchor portion comprising a first anchor and a second anchor, wherein the first and second anchors are configured to be moved from an open position to a closed position to secure the implantable device to leaflets of a native valve;
one or more leak control extensions configured to inhibit blood regurgitation through one or more openings between leaflets of the native valve during systole; and
wherein the one or more leak control extensions are configured to be compressed into a deformed shape upon being pressed against an annulus of the native valve or a heart wall.

2. The valve repair device according to claim 1, wherein the compressed leak control extensions are configured to cover a deformed opening of the native heart valve that is adjacent to the annulus.

3. The valve repair device according to claim 1, further comprising a spacer and wherein the first anchor and the second anchor are disposed on opposite sides of the spacer.

4. The valve repair device according to claim 3, wherein the one or more leak control extensions comprise a first leak control extension and a second leak control extension, the first leak control extension and the second leak control extension are disposed on opposite sides of the spacer, and the first leak control extension and the second leak control extension are each disposed between the first anchor and the second anchor.

5. The valve repair device of claim 1 wherein the one or more leak control extensions comprise one or more pieces of a cloth material.

6. The valve repair device according to claim 1, wherein the barrier element comprises one or more pieces of a biocompatible material.

7. The valve repair device according to claim 1, wherein each of the leak control extensions comprises one or more deflecting paddles.

8. The valve repair device according to claim 7, wherein the deflecting paddle comprises a wire frame with a cloth covering.

9. A method of repairing a native valve of a patient, the method comprising:
positioning a heart valve repair device in the native heart valve and pressing a leak control extension of the heart valve repair device against an annulus or a heart wall to compress the leak control extension into a deformed shape;
moving a first anchor of the valve repair device from an open position to a closed position to secure the valve repair device to a first leaflet of the native valve; and
moving a second anchor of the valve repair device from an open position to a closed position to secure the valve repair device to a second leaflet of the native valve.

10. The method of claim 9 further comprising blocking blood regurgitation through an opening between the first leaflet and the second leaflet of the native valve during ventricular systole.

11. The method according to claim 9, further comprising covering a deformed opening of the native heart valve that is adjacent to the annulus with the compressed leak control extension.

12. The method according to claim 9, wherein the valve repair device comprises a spacer and wherein the first anchor and the second anchor are disposed on opposite sides of the spacer.

13. The method according to claim 12, wherein the leak control extension is disposed between the first anchor and the second anchor.

14. The method according to claim 9, wherein each of the leak control extensions comprise one or more deflecting paddles.

15. The method according to claim 14, wherein the deflecting paddle comprises a wire frame with a cloth covering.

16. A valve repair device for repairing a native valve of a patient, the valve repair device comprising:
an anchor portion comprising a first anchor and a second anchor, wherein the first and second anchors are configured to be moved from an open position to a closed position to secure the implantable device to leaflets of a native valve; and
one or more leak control extensions configured to inhibit blood regurgitation through one or more openings between leaflets of the native valve during systole, wherein the leak control extension comprises a barrier element attached to the first and second anchors.

17. The valve repair device according to claim 16, wherein the barrier element extends from the first anchor to the second anchor to create a barrier that extends between the first anchor and the second anchor.

18. The valve repair device according to claim 16, wherein each of the first and second anchors comprise a paddle frame, and wherein the barrier element is attached to the paddle frame of both the first and second anchors.

19. The valve repair device according to claim 16, wherein each of the leak control extensions comprise one or more deflecting paddles.

20. The valve repair device according to claim 19, wherein the barrier element connects to and extends from the first anchor, two deflecting paddles of the one or more deflecting paddles, and to the second anchor.
